(19) United States
(12) Patent Application Publication
(10) Pub. No.: US 2024/0018470 A1
(43) Pub. Date: Jan. 18, 2024

(54) METHOD OF MANIPULATING PLURIPOTENCY IN CELLS

(71) Applicant: YALE UNIVERSITY, New Haven, CT (US)

(72) Inventors: Valentyna Kostiuk, New Haven, CT (US); Mustafa Khokha, New Haven, CT (US)

(21) Appl. No.: 18/350,978

(22) Filed: Jul. 12, 2023

Related U.S. Application Data
(60) Provisional application No. 63/388,380, filed on Jul. 12, 2022.

(51) Int. Cl. C12N 5/0735

(52) U.S. Cl. CPC C12N 5/0606; C12N 2500/12; C12N 2501/998

(57) ABSTRACT

Described herein is a method of maintaining pluripotency in a cell. The method includes in one aspect reducing a membrane potential of the cell. Also described herein is a culture medium for performing certain aspects of the method, as well as a composition in which the pluripotency of a cell is maintained by the culture medium.

### CROSS-REFERENCE TO RELATED APPLICATIONS

The present application claims priority under 35 U.S.C. § 119(e) to U.S. Provisional Patent Application No. 63/388,380, filed Jul. 12, 2022, which is incorporated herein by reference in its entirety.

### STATEMENT REGARDING FEDERALLY SPONSORED RESEARCH

This invention was made with government support under 1HL149746-01A1 awarded by National Institutes of Health. The government has certain rights in the invention.

### SEQUENCE LISTING

The ASCII text file named “047162-7368US1_SeqListing.xml” created Jul. 11, 2023, comprising 17.4 Kbytes, is hereby incorporated by reference in its entirety.

### BACKGROUND

During certain stages of the embryonic development, cells need to extinguish pluripotency factors so as to activate pathways of cellular differentiation. The understanding of the factors controlling this step would allow the manipulation of pluripotency in cells, for example the ability to maintain pluripotency in stem cells. Therefore, there is a need to elucidate factors that control the exit of cells from pluripotency. This knowledge would allow for rationally manipulating pluripotency in cells. The present invention addresses this need.

### SUMMARY

In some aspects, the present invention is directed to the following non-limiting embodiments.

In some embodiments, the present invention is directed to a method of maintaining pluripotency in a cell.

In some embodiments, the method comprises at least one of the following: reducing the membrane potential of the cell; activating a voltage gated calcium channel on the plasma membrane of the cell; and/or increasing the calcium ion concentration in the cell.

In some embodiments, reducing the membrane potential of the cell comprises at least one of the following: subjecting the cell to an extracellular environment having a high concentration of potassium ions; inhibiting a potassium channel on the plasma membrane of the cell; and/or contacting the cell with a potassium selective ionophore.

In some embodiments, reducing the membrane potential of the cell comprises subjecting the cell to an extracellular environment having a potassium ion concentration of about 0.5 mM or higher, such as about 0.75 mM or higher, about 1 mM or higher, about 2 mM or higher, about 3 mM or higher, about 4 mM or higher, about 5 mM or higher, about 7.5 mM or higher, about 10 mM or higher, about 12.5 mM or higher, about 15 mM or higher, about 20 mM or higher, about 25 mM or higher, about 30 mM or higher, about 40 mM or higher, or about 50 mM or higher.

In some embodiments, reducing the membrane potential of the cell comprises inhibiting a potassium channel on the plasma membrane of the cell, wherein the potassium channel comprises an inwardly-rectifying voltage gated potassium channel.

In some embodiments, the inwardly-rectifying voltage gated potassium channel comprises potassium voltage-gated channel subfamily H member 6 (KCNH6).

In some embodiments, inhibiting the potassium channel on the plasma membrane of the cell comprises contacting the cell with a potassium channel inhibitor.

In some embodiments, the inhibitor of the potassium channel comprises barium ions or an Ergtoxin.

In some embodiments, reducing the membrane potential of the cell comprises contacting the cell with a potassium selective ionophore comprising valinomycin, BME 44 (2-Dodecyl-2-methyl-1,3-propanediyl bis[N-[5′-nitro(benzo-15-crown-5)-4′-yl]carbamate]), or BB15C5 (Bis[(benzo-15-crown-5)-4′-ylmethyl] pimelate).

In some embodiments, the voltage gated calcium channel is an L-type calcium channel or a T-type calcium channel.

In some embodiments, the cell is a stem cell.

In some embodiments, the cell is an embryonic stem cell.

In some embodiments, the cell is in an organism, a cultured primary cell, or a cultured cell line.

In some embodiments, the cell is from a vertebrate origin.

In some embodiments, the cell is from a mammalian origin.

In some embodiments, the cell is from a human origin.

In some aspects, the present invention is directed to a composition.

In some embodiments, the composition comprises a culture medium and a pluripotent cell.

In some embodiments, potassium ions in the culture medium are present in a concentration of about 0.5 mM or higher.

In some embodiments, a pluripotency of the pluripotent cell is maintained by the concentration of potassium ions in the culture medium.

In some embodiments, the concentration of the potassium ions in the culture medium reduces the membrane potential of the cell, thereby maintaining the pluripotency of the pluripotent cell.

In some embodiments, the culture medium comprises: at least one inorganic ion selected from a sodium ion, a potassium ion, a calcium ion, a magnesium ion, a chloride ion, a sulfate ion, a carbonate ion, a bicarbonate ion, a phosphate ion, a phosphate monobasic ion, a phosphate dibasic ion; an amino acid; and a vitamin.

In some embodiments, the amino acid comprises at least one selected form the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

In some embodiments, the vitamin comprises at least one selected from the group consisting of pantothenate, choline, folic acid, inositol, nicotinamide, pyridoxine, riboflavin, and thiamine.

In some embodiments, the culture medium further comprises a carbohydrate.

In some embodiments, the culture medium further comprises one or more selected form the group consisting of pyruvate, lipoic acid, biotin, a buffering agent, and a pH indicator.

In some embodiments, the pluripotent cell is a stem cell or an embryonic stem cell.

In some embodiments, the pluripotent cell is from a vertebrate origin.

In some embodiments, the pluripotent cell is from a mammalian origin.

In some embodiments, the pluripotent cell is from a human origin.

In some embodiments, the pluripotent cell does not have the cell potency to develop in to a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1Q demonstrate that membrane potential is important for gastrulation and LR patterning, in accordance with some embodiments. FIG. 1A: GHK equation for Vₘ; R=gas constant, T=temperature, F=Faraday's constant, p=permeability for each ion, [X]o=ion concentration outside of cell, [X]i=ion concentration inside the cell. FIGS. 1B-1I: Effects of depolarizing treatments (barium chloride and high K⁺) and kcnh6 depletion on gastrulation (FIGS. 1B-1E; stage 15-17 embryos; arrowheads indicate incomplete blastopore closure) and organ situs (FIGS. 1F-1I; stage 45 tadpoles; ventral views; arrowheads indicate normal (D) and inverse (L) heart looping). FIG. 1J: Different stages of barium chloride application and effects on gastrulation and LR patterning (color key in FIG. 1J for bar graphs FIG. 1K and FIG. 1L); green=cleavage stages (stages 0-6 or 0-8); red=gastrulation (stages 8-12); blue=LRO signaling (stages 12-19); orange=early organogenesis (stages 19-30); grey=cleavage through LRO signaling (stages 0-19). FIGS. 1K-1L: Percentages of embryos with incomplete blastopore closure at stage 15 (FIG. 1K) and abnormal organ situs at stage 45 (FIG. 1L) after treatment with barium at different stages (see (FIG. 1J)). FIG. 1M: Percentages of embryos with abnormal gastrulation after depletion of kcnh6 (MO, CRISPR) or Kcnh channel blockade with Ergtoxin, and rescue of kcnh6 depletion with medium conditions that hyperpolarize the Vₘ (low K⁺; val=valinomycin; sodium substitution with choline; all treatments performed from stage 8 to stage 12); on the left: examples of embryos scored for the graph; posterior views (dorsal to the top) of stage 15 embryos after successful (control) or unsuccessful (kcnh6 CRISPR) gastrulation; arrowhead points to blastopore closure. FIG. 1N: Representative intracellular recording in the prospective ectoderm of a control stage 10 embryo; Vₘ is measured relative to the medium (baseline); the dip in membrane potential indicates the electrode breaking into the cell. The graph displays the Vₘ as measured in the prospective ectoderm of stage 10 Control MO vs kcnh6 MO injected embryos; each data point represents one cell; data are from three independent experiments. FIG. 1O: Live animal pole images of GCaMP6/mCherry at stage 10. FIG. 1P: Quantification of GCaMP6 fluorescence intensity normalized to mCherry in mCherry+ cells; data points represent single cells. FIG. 1Q: Maximum area undergoing simultaneous Ca²⁺ transients within a 20 s time lapse recording as a percentage of total animal pole area.

FIGS. 2A-2O demonstrate that membrane potential affects early gastrula patterning and pluripotency, in accordance with some embodiments. FIGS. 2A-2J: WMISH for germ layer markers in early gastrula embryos (stage 10; FIGS. 2A and 2C, vegetal views with dorsal to the top; FIGS. 2E, 2G and 2I: lateral view of bisected embryos, dorsal to right). Markers are for paraxial mesoderm (myoD), superficial dorsal mesoderm (foxj1), ectoderm (ectodermin), endoderm (vegT, mixer). FIGS. 2K-2N: WMISH for Xenopus pluripotency genes at stages 9 and 10 (animal pole views). FIG. 2O: Differentiation potential of animal caps excised at stage 8 or 12 and treated with no activin (differentiation into epidermis marked by cytokeratin), low activin (differentiation into mesoderm marked by tbxt) and high activin (differentiation into endoderm marked by sox17β). (*=p≤0.05, ***=p≤0.001, ****=p≤0.0001, ns=non-significant; error bars: ±SEM).

FIGS. 3A-3J illustrate the role of VGCCs in germ layer differentiation, in accordance with some embodiments. FIGS. 3A-3D: WMISH for ectodermin; lateral views with the animal pole to the top; asterisk marks the animal pole with loss of ectodermin expression. FIGS. 3E-3H: WMISH for myf5: vegetal views with dorsal to the top; arrowhead marks loss of expression. FIG. 3I: Quantification of abnormal (absent) expression of ectodermin in FIGS. 3A-3D. FIG. 3J: Quantification of abnormal expression of FIGS. 3E-3H. CR (CRISPR), Nfd (nifedipine). (*=p≤0.05, **=p≤0.01, ***=p≤0.001, ****=p≤0.0001; ns=non-significant; error bars: ±SEM).

FIGS. 4A-4E: High resolution temporal RNAseq identifies mTOR and Ca²⁺ GRN, in accordance with some embodiments. FIGS. 4A-4B: Summary of activated gene clusters by (FIG. 4A) heatmap and (FIG. 4B) cluster average. Data is Gaussian process median for each gene normalized by maximal value, shaded region in FIG. 4B is ±1 SD for each cluster. UIC=Uninjected Control (U); High K⁺ (depolarizing conditions, H or HiK). FIG. 4C: Bubble plot for selection of gene set enrichment terms, calculated with Enrichr, see Methods for definition of terms. Bubble size reflects Enrichr Combined score and color indicates −log 10 FDR. FIG. 4D: Bubble plot enrichment of TF motifs in 500 bp upstream of cluster promoters, see also FIG. 18G. Bubble size reflects fold change over background and color is −log 10 p-value for enrichment. FIG. 4E: Expression of exemplar genes in control and high K⁺. Smooth line and shaded region are transformed Gaussian process median and 95% CI. Circle in top right hand corner gives cluster number. UIC (uninjected control) High K⁺ (depolarizing conditions).

FIGS. 5A-5F demonstrate that Vₘ polarization limits mTOR which promotes pluripotency, in accordance with some embodiments. FIG. 5A: WMISH for pou5f3.3 (top row, animal pole view, expression quantitated in (FIG. 5D)), FIG. 5B: ventx1.2 (lateral view with dorsal to the right, expression quantitated in (FIG. 5E)), and FIG. 5C: ectodermin (lateral view with dorsal to the right, expression quantitated in (FIG. 5F)) of stage 10 embryos. Embryos are treated with vehicle (DMSO) or rapamycin as indicated. CR (CRISPR), hiK (high K⁺/depolarizing conditions), Rapa (rapamycin), AP (animal pole), VMZ (ventral marginal zone). (±) in FIGS. 5D-5E indicate presence or absence, respectively, of gene expression. (****=p≤0.0001; error bars: ±SEM).

FIGS. 6A-6E demonstrate that potassium channels affect pluripotency in hESCs, in accordance with some embodiments. FIG. 6A: Images showing untreated hESCs grown in mTeSR1 media or cells treated with 1 mM Barium or 25 nM Ergtoxin and immunostained for pluripotency factors. FIG. 6B: Quantification of the results in FIG. 6A; AU=arbitrary units. (*=p≤0.05, **=p≤0.01; error bars: ±SEM). FIG. 6C: Images showing untreated hESCs grown in MEF-CM media or cells treated with 100 nM rapamycin with or without 25 nM Ergtoxin. FIG. 6D: Quantification of the results in FIG. 6C; AU=arbitrary units. (*=p≤0.05, **=p≤0.01; ***=p<0.001; error bars: ±SEM). FIG. 6E: Model for the onset of embryonic differentiation depicting classical biochemical signaling (i) that is complemented by regulation via membrane potential (ii). In the electrophysiological pathway, potassium channels set the membrane potential, which limits activation of voltage-gated calcium channels and suppresses intracellular Ca²⁺ levels. Both pathways result in changes in gene expression, mediated by intracellular signal transducers (i), or by factors that require calcium (ii). While biochemical pathways are essential to induce expression of differentiation factors, the electrophysiological pathway affects cell fate indirectly by controlling the timing of downregulation of pluripotency genes. (*=p≤0.05, **=p≤0.01, ***=p≤0.001, 472 ****=p≤0.0001; ns=non-significant; error bars: ±SEM).

FIG. 7: KCNH gene variants identified in patients with CHD, in accordance with some embodiments. CTD=conotruncal defect; Htx=heterotaxy; LVO=left ventricular outflow tract obstruction; TGA=transposition of the great arteries; LOF=loss of function; CmpHet=compound heterozygote, misD=damaging missense mutation.

FIG. 8: Antibodies used in the present study.

FIG. 9: Meanings of some terms used herein.

FIGS. 10A-10I: WMISH expression analysis for kcnh6 during Xenopus development, in accordance with some embodiments. FIGS. 10A-10H: kcnh6 transcripts were detected by a full-length antisense probe with the following orientations: FIG. 10A, animal pole to the top, FIG. 10B, animal pole view FIG. 10C, bisected with animal pole to the top, FIG. 10D, animal pole view, FIG. 10E: vegetal view and dorsal to the top, FIG. 10F, gastrocoel roof plate with anterior to the top and vegetal view, FIGS. 10G and 10H, lateral view with anterior to the left and dorsal to the top Ecto=prospective ectoderm; Endo=prospective endoderm; DMZ=dorsal marginal zone (mesoderm); PM=paraxial mesoderm; som=somites; GRP=gastrocoel roof plate. FIG. 10I: kcnh6 transcripts (high temporal resolution RNA-seq3) and Vₘ⁶ (black) plotted during early Xenopus development.

FIGS. 11A-11L demonstrate that kcnh6 depletion or Ergtoxin induces LR patterning defects, in accordance with some embodiments. FIGS. 11A-11E: Examples of organ situs in stage 47 X. tropicalis tadpoles (ventral view with anterior to the top). Arrowheads indicate normal (D) and abnormal (L and A; A=outflow tract is midline) cardiac looping; the heart and gall bladder are minimally pseudocolored in pink and green respectively for visualization; asterisks indicate the location of the liver whenever discernible. FIGS. 11F-11H: Quantification of stage 47 tadpoles with situs defects upon injection of CRISPRs targeting independently two sites in different exons of the kcnh6 locus, translation blocking MO, or after treatment with Ergtoxin. For all graphs, tadpoles with multiple defects were counted only once. FIGS. 11I-11J: Detection of dand5 expression via WMISH in stage 16 (pre-ciliary flow) and stage 19 (post flow) embryos viewed ventrally (FIG. 111; A=anterior, P=posterior; R=right, L=left); the quantification in FIG. 11J indicates percentages of embryos with absent dand5 expression. FIGS. 11K-11L: Detection of pitx2c expression in stage 28 embryos via WMISH; embryos in FIG. 11K are lateral views with dorsal to the top and either the left (L) or right (R) side visible; the quantification in FIG. 11L indicates percentages of embryos with abnormal (absent or bilateral) pitx2c expression. (*=p≤0.05, **=p≤0.01, ***=p≤0.001, ****=p≤0.0001; ns=non-significant; error bars: ±SEM).

FIGS. 12A-12D: Inference of Crispr Edits (ICE) analysis in kcnh6 CRISPR stage 47 tadpoles, in accordance with some embodiments. FIGS. 12A and 12C: Average percentages of frameshift (KO=knockout), in-frame (other) or unedited kcnh6 (no edits) sequences for CRISPRs targeting exon 3 (FIG. 12A) or exon 4 (FIG. 12C). FIGS. 12B and 12D: Distribution of INDEL sizes; INDELs with representation >5% of total sequences are represented in individual bars, whereas those with <5% are summed in the last bar (*). All graphs display averages of 14 tadpoles per CRISPR from two independent experiments (±SEM).

FIGS. 13A-13B: Controls to FIG. 1M and Vₘ measurements of High K⁺ and choline embryos, in accordance with some embodiments. FIG. 13A: effects of KCNH6 mRNA expression and hyperpolarizing conditions on gastrulation in control or kcnh6 MO embryos. Percentages of embryos with incomplete blastopore closure at stage 17. FIG. 13B: Intracellular recordings in the prospective ectoderm of stage 9 embryos; Vₘ was measured relative to the medium; each data point represents one cell; cholineCl refers to substitution of ½Na⁺ with choline (*=p≤0.05, **=p≤0.01, ***=p≤0.001, ****=p≤0.0001; error bars: ±SEM).

FIGS. 14A-14T: Effects of kcnh6 depletion and Vₘ depolarization on germ layer differentiation, in accordance with some embodiments. FIGS. 14A-14F: WMISH for mesodermal transcripts. FIGS. 14A-14B: vegetal views with dorsal to the top of paraxial mesodermal markers myod, myf5. FIG. 14C: quantitation of abnormal expression of myf5. FIG. 14D: lateral views of tbxt (except under high K⁺ which is vegetal). * marks break in mesoderm in paraxial region that is quantitated in FIG. 14E. FIG. 14F: vegetal views with dorsal to the top. FIGS. 14G-14L WMISH for transcripts of dorsal organizer (gsc, xnr3) and ventral mesoderm (vent2) marker genes; vegetal views of stage 10 embryos with dorsal to the top. FIGS. 14M-14R: WMISH for transcripts of the ectoderm or endoderm. Bisected embryos with animal pole to the top and dorsal to the right. FIGS. 14S-14T: WMISH for foxIla, expressed in the prospective ectoderm; animal views of stage 10 embryos. All graphs show percentages of embryos with abnormal (absent) expression (****=p≤0.0001, ***=p≤0.001, ns=non-significant; ±SEM).

FIG. 15 illustrates the effects of kcnh6 depletion on LRO patterning, in accordance with some embodiments. Immunostaining of the LRO in stage 17 embryos for acetylated tubulin (cilia) and MyoD (paraxial mesoderm); cell morphology is highlighted with phalloidin (actin); ventral views with anterior to the top; arrowheads indicate areas of absent MyoD labelling in the paraxial margins of the LRO in kcnh6 CRISPR embryos.

FIGS. 16A-16D: Pou5f3.2 and the role of VGCCs in pluripotency, in accordance with some embodiments. FIG. 16A: animal pole view; no differences in expression of pou5f3.2 between control and kcnh6 CR (CRISPR) noted as quantitated in right panel. FIG. 16B: lateral views with animal pole to the top. Nfd (nifedipine). WMISH is quantitated in FIG. 16C for pou5f3.3 and in FIG. 17D for ventx1.2. (*=p≤0.05, **=p≤0.01, ***=p≤0.001, ****=p≤0.0001; ns=non-significant; error bars: ±SEM).

FIGS. 17A-17J: High Temporal Resolution RNAseq, in accordance with some embodiments. FIG. 17A: Schematic of RNAseq collection from stage 8 to stage 12. 10 embryos were collected every 30 mins from a clutch of synchronized embryos after IVF. FIG. 17B: Quantification of ERCC spike-in transcripts before (top) and after (bottom) dinucleotide correction to account for GC bias for UIC=Uninjected Control and High K⁺ embryos. Note that true spike-in concentration is on the horizontal axis, and spikes with low GC content are underrepresented in sequencing. Corrected quantifications are used in all subsequent panels. FIG. 17C: Pairwise Spearman correlation between all UIC and High K⁺ samples after filtering for genes with sufficient temporal expression. FIG. 17D: Principal components analysis of log(TPM+1) transformed expression for UIC and HK⁺=High K⁺ samples. Sample index 1-13 labelled. FIG. 17E: Visualisation of total differentially expressed genes and their magnitude as 2D histogram, colour indicating the frequency of genes in each bin. Horizontal axis gives log-likelihood-ratio, with LR>0 defined as differentially expressed. Vertical axis gives maximal divergence z-score of UIC and High K⁺ trajectories in transformed GP space, averaging signal and noise variance for UIC and High K⁺. FIGS. 17F-17G: K-means clustering of activated and repressed genes as (FIG. 17F) heatmap and (FIG. 17G) cluster average. Shaded region in FIG. 17F gives ±1 SD for each cluster as in FIG. 3B. FIG. 17H: Top 16 motif enrichments promoters (500 bp upstream of TSS) of activated clusters A1, A2, A3, A4. Heatmap gives −log 10 p-value sorted by maximal enrichments such that motifs enriched in A1/A2 are at the top and those in A4 are at the bottom. FIG. 17I: Expression of calcium responsive TFs, ets1, crem, creb1, atf1, demonstrating that these genes are not transcriptionally activated over the timecourse. Log-likelihood ratio given with LR>0 differentially expressed. FIG. 17J: Enrichments of public ChIP-seq peaks for CREB1, CREM and ETS in tissues and cell lines given in Human, Mouse and Rat, in proximity to genes in activated clusters A1, A2, A3, A4. Enrichments calculated with Enrichr and ChEA_2016 gene set7 which integrates data from a broad range of publicly available ChIP assays. ChEA_2016 term names given on horizontal axis describing target, Pubmed ID, cell/tissue and organism. Left panel gives size of intersection as percent of total genes in each cluster, right panel gives −log 10 FDR for Fisher's Exact test for overrepresentation in each cluster.

FIGS. 18A-18F demonstrate that blocking KCNH channels slows differentiation of hESCs while rapamycin treatment induces differentiation, in accordance with some embodiments. FIGS. 18A-18B: Cells were grown with or without ErgToxin reagent (25 nM) for 2 (FIG. 18A) or 5 days (FIG. 18B) and then the indicated pluripotency markers were measured by qRT-PCR. FIGS. 18C-18D: Quantification of immunofluorescence against SOX2 and NANOG following treatment with 50 ng/ml BMP4 with or without Ergtoxin addition. (AU=arbitrary units). FIG. 18E: Expression of pluripotency markers OCT4, SOX2 and NANOG in hESCs (relative immunofluorescence intensity, arbitrary units) upon treatment with rapamycin alone, or rapamycin and ErgToxin together, after seeding at different densities. 1000, 3000, or 7000 cells were seeded into an 18 well slide (Ibidi) and treated with rapamycin (100 nM) for 5 days. As a control, 1000 cells were seeded and grown without rapamycin treatment. FIG. 18F: Pluripotency marker expression data from FIG. 18E plotted as a function of final cell density. Numbers indicated for final density are the average number of cells in one image, whose dimensions correspond to 0.636 mm×0.636 mm. Pluripotency markers are reduced in the rapamycin treated cells regardless of whether the final density is higher or lower than in the control condition.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

The study described herein (“the present study”), using xenopus embryos (such as embryos at blastula or gastrula stages) and human embryonic stem cells (hESCs) as non-limiting examples, discovered that pluripotency of cells, such as embryonic cells or stem cells, can be maintained by reducing the cellular membrane potentials (i.e., depolarizing the cells).

Accordingly, in some aspects, the present invention is directed to a method of maintaining pluripotency in a cell.

The present study further discovered that, one way to depolarize cell membranes and maintain pluripotency in the cell is to culture the cell in a medium having higher than usual potassium ion concentrations.

Accordingly, in some aspects, the present invention is directed to a culture medium for maintaining pluripotency in cultured cells.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

### Method of Maintaining Cell Pluripotency

The present study, using xenopus embryos (such as embryos at blastula or gastrula stages) and human embryonic stem cells (hESCs) as non-limiting examples, discovered that pluripotency of cells, such as embryonic cells or stem cells, can be maintained by reducing the membrane potential of the cells.

Accordingly, in some aspects, the present specification is directed to a method of maintaining pluripotency in a cell.

In some embodiments, the method includes reducing the cell membrane potential; activating a voltage gated calcium channel in the cell membrane (plasma membrane); and/or increasing calcium ion concentration in the cell.

As used herein, the term “reducing membrane potential” means bringing the membrane potential of the cell closer to 0. For example, if a membrane potential of the cell is about −70 mV, the membrane potential is considered to be reduced if the membrane potential is brought to about −60 mV, −50 mV, −40 mV, −30 mV, and/or −20 mV. The present specification sometimes refers to “reducing membrane potential” of a cell as “depolarizing” the membrane of the cell.

In some embodiments, reducing the membrane potential of the cell includes: subjecting the cell to an extracellular environment having a high concentration of potassium ions; inhibiting a potassium channel in a plasma membrane of the cell; or contacting the cell with a potassium selective ionophore.

In some embodiments, subjecting the cell to an extracellular environment having a high concentration of potassium ions includes subjecting the cell to an extracellular environment having a concentration of potassium ions of about 0.5 mM or higher, such as about 0.75 mM or higher, about 1 mM or higher, about 2 mM or higher, about 3 mM or higher, about 4 mM or higher, about 5 mM or higher, about 7.5 mM or higher, about 10 mM or higher, about 12.5 mM or higher, about 15 mM or higher, about 20 mM or higher, about 25 mM or higher, about 30 mM or higher, about 40 mM or higher, or about 50 mM or higher. In some embodiments, subjecting the cell to an extracellular environment having a high concentration of potassium ions includes culturing the cell in a culture medium having an alleviated potassium ion concentration, such as the concentrations described herein.

In some embodiments, reducing the membrane potential of the cell includes inhibiting the potassium channel on the plasma membrane of the cell. In some embodiments, the potassium channel comprises an inwardly-rectifying voltage gated potassium channel. In some embodiments, the inwardly-rectifying voltage gated potassium channel includes potassium voltage-gated channel subfamily H member 6 (KCNH6).

In some embodiments, inhibiting the potassium channel on the plasma membrane of the cell comprises contacting the cell with an inhibitor of the potassium channel. In some embodiments, the inhibitor of the potassium channel includes barium ions or an Ergtoxin.

In some embodiments, reducing the membrane potential of the cell comprises contacting the cell with a potassium selective ionophore. In some embodiments, the potassium selective ionophore comprises valinomycin, BME 44 (2-Dodecyl-2-methyl-1,3-propanediyl bis[N-[5′-nitro(benzo-15-crown-5)-4′-yl]carbamate]), or BB15C5 (Bis[(benzo-15-crown-5)-4′-ylmethyl] pimelate).

In some embodiments, the calcium channel is an L-type calcium channel or a T-type calcium channel.

In some embodiments, the cell which pluripotency is maintained by the method is a stem cell. In some embodiments, the cell is an embryonic stem cell. In some embodiment the cell is in an organism, a cultured primary cell, or a cultured cell line. In some embodiments, the cell is from a vertebrate origin, such as from a mammalian origin, or from a human origin.

### Culture Medium for Maintaining Pluripotency

The present study discovered that one way to depolarize cell membranes and maintain pluripotency in the cell is to culture the cell in a culture medium having higher than usual potassium ion concentrations.

Accordingly, in some aspects, the present specification is directed to a culture medium. In some embodiments, the culture medium is for maintaining a pluripotency of a cell cultured therein.

In some embodiments, the culture medium includes at least one of the following:
inorganic ions including one or more of sodium ions, potassium ions, calcium ions, magnesium ions, chloride ions, sulfate ions, carbonate/bicarbonate ions, and phosphate/phosphate monobasic/phosphate dibasic ions;
one or more amino acids; and/or
one or more vitamins.

In some embodiments, the concentration of potassium ions in the culture medium is about 0.5 mM or higher, such as about 0.75 mM or higher, about 1 mM or higher, about 2 mM or higher, about 3 mM or higher, about 4 mM or higher, about 5 mM or higher, about 7.5 mM or higher, about 10 mM or higher, about 12.5 mM or higher, about 15 mM or higher, about 20 mM or higher, about 25 mM or higher, about 30 mM or higher, about 40 mM or higher, or about 50 mM or higher.

In some embodiments, the one or more amino acids include at least one of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or combinations thereof. In some embodiments, some of the amino acids are L-amino acids. In some embodiments, all of the amino acids are L-amino acids.

In some embodiments, the one or more vitamins include pantothenate, choline, folic acid, inositol, nicotinamide, pyridoxine, riboflavin, thiamine, or combinations thereof.

In some embodiments, the culture medium further includes one or more carbohydrates. In some embodiments, the one or more carbohydrates include D-glucose.

In some embodiments, the culture medium further includes one or more of pyruvate, lipoic acid, biotin, a buffering agent (such as HEPES), a pH indicator (such as phenol red).

In some embodiments, the culture medium is the same as or similar to existing culture media, such as RPMI 1640 (Moore et al, JAMA. 1967 Feb. 20; 199(8):519-24.), Iscove's Modified Dulbecco's Medium (IMDM), Minimum Essential Medium Eagle (MEM) (Eagle, Science. 1959 Aug. 21; 130(3373):432-7) or Dulbecco's Modified Eagle Medium (DMEM) (Dulbecco et al., Virology, Volume 8, Issue 3, July 1959, Pages 396-397), except for the alleviated concentrations of potassium ions. In some embodiments, the concentration of each of the non-potassium components is within about ±20%, such as about ±15%, about ±12.5%, about ±10%, about ±7.5%, about ±5%, about ±2% or about ±1% of the currently used concentrations.

In some embodiments, the cell which pluripotency is maintained by the culture media is a stem cell. In some embodiments, the cell is an embryonic stem cell. In some embodiment the cell is in an organism, a cultured primary cell, or a cultured cell line. In some embodiments, the cell is from a vertebrate origin, such as from a mammalian origin, or from a human origin.

### Composition Including Pluripotent Cell

The present study discovered that keeping pluripotent cells in in a culture medium having higher than usual potassium ion concentrations is able to maintain the pluripotency in the cell.

Accordingly, in some aspects, the present invention is directed to a composition including a pluripotent cell. In some embodiments, other components in the composition maintains the pluripotency of the pluripotent cell.

In some embodiments, the composition includes a pluripotent cell and a culture medium. In some embodiments, the culture medium is the same as or similar to those as described elsewhere herein, such as in the “Culture Medium for Maintaining Pluripotency” section.

In some embodiments, the pluripotent cell is a stem cell. In some embodiments, the pluripotent cell is an embryonic stem cell. In some embodiment the pluripotent cell is in an organism, a cultured primary cell, or a cultured cell line. In some embodiments, the pluripotent cell is from a vertebrate origin, such as from a mammalian origin, or from a human origin. In some embodiments, the pluripotent cell does not have the cell potency to develop in to a human.

### EXAMPLES

The present specification further describes in detail by reference to the following experimental examples. These examples are provided for purposes of illustration only, and are not intended to be limiting unless so specified. Thus, the present specification should in no way be construed as being limited to the following examples, but rather, should be construed to encompass any and all variations which become evident as a result of the teaching provided herein.

### Example 1

Transitioning from pluripotency to differentiated cell fates is fundamental to both embryonic development and adult tissue homeostasis. Improving the understanding of this transition would facilitate the ability to manipulate pluripotent cells into tissues for therapeutic use.

Referring to the Example 2 section herein, the study described herein (“the present study”) demonstrates that membrane voltage (Vₘ) regulates the exit from pluripotency and the onset of germ layer differentiation in the embryo, a process that affects both gastrulation and left-right patterning. By examining candidate genes of congenital heart disease and heterotaxy, KCNH6, a member of the ether-a-go-go class of potassium channels that hyperpolarizes the Vₘ and thus limits the activation of voltage gated calcium channels, lowering intracellular calcium, was identified. In pluripotent embryonic cells, depletion of kcnh6 led to membrane depolarization, elevated intracellular calcium levels, and the maintenance of a pluripotent state at the expense of differentiation into ectodermal and myogenic lineages. Using high-resolution temporal transcriptome analysis, the present study identifies the gene regulatory networks downstream of membrane depolarization and calcium signaling and discover that inhibition of the mTOR pathway transitions the pluripotent cell to a differentiated fate. By manipulating Vₘ using a suite of tools, the present study establishes a bioelectric pathway that regulates pluripotency in vertebrates, including human embryonic stem cells.

### Example 2: Membrane Potential Drives the Exit from Pluripotency and the Ontogeny of Cell Fate Via Calcium and mTOR

Transitioning from pluripotency to differentiated cell fates is fundamental to both embryonic development and adult tissue homeostasis. Improving the understanding of this transition would facilitate the ability to manipulate pluripotent cells into tissues for therapeutic use. Here, the present study shows that membrane voltage (Vₘ) regulates the exit from pluripotency and the onset of germ layer differentiation in the embryo, a process that affects both gastrulation and left-right patterning. By examining candidate genes of congenital heart disease and heterotaxy, the present study identified KCNH6, a member of the ether-a-go-go class of potassium channels that hyperpolarizes the Vₘ and thus limits the activation of voltage gated calcium channels, lowering intracellular calcium. In pluripotent embryonic cells, depletion of kcnh6 led to membrane depolarization, elevated intracellular calcium levels, and the maintenance of a pluripotent state at the expense of differentiation into ectodermal and myogenic lineages. Using high-resolution temporal transcriptome analysis, the present study identified the gene regulatory networks downstream of membrane depolarization and calcium signaling and discover that inhibition of the mTOR pathway transitions the pluripotent cell to a differentiated fate. By manipulating Vₘ using a suite of tools, the present study established a bioelectric pathway that regulates pluripotency in vertebrates, including human embryonic stem cells.

### Example 2-1

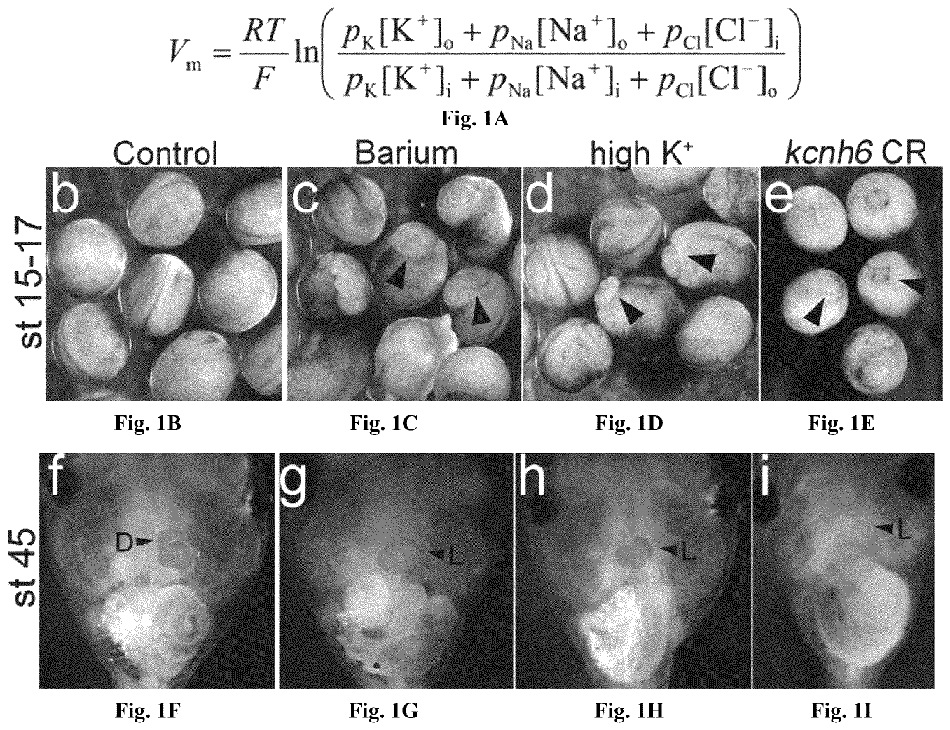

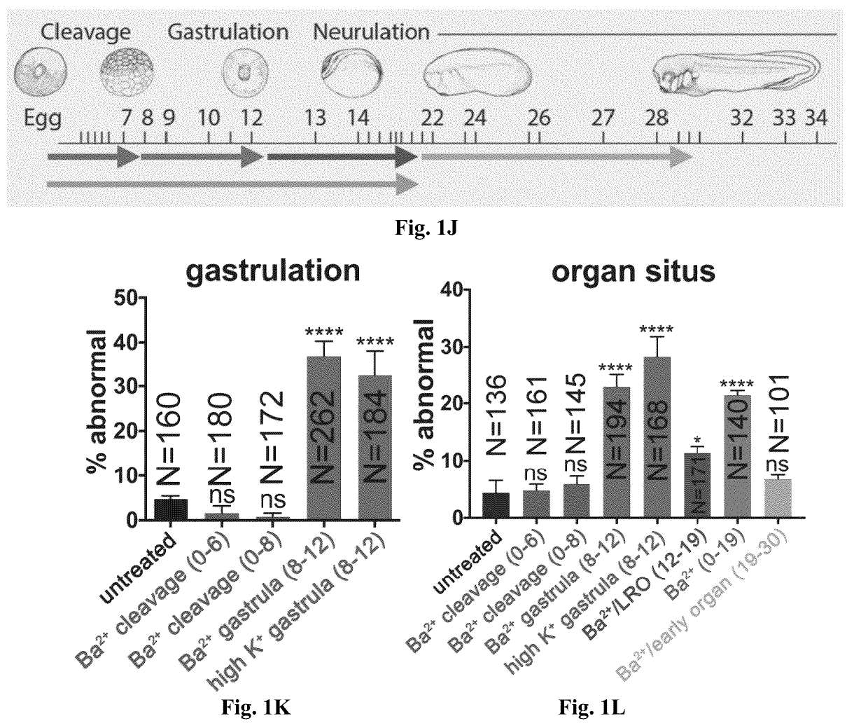

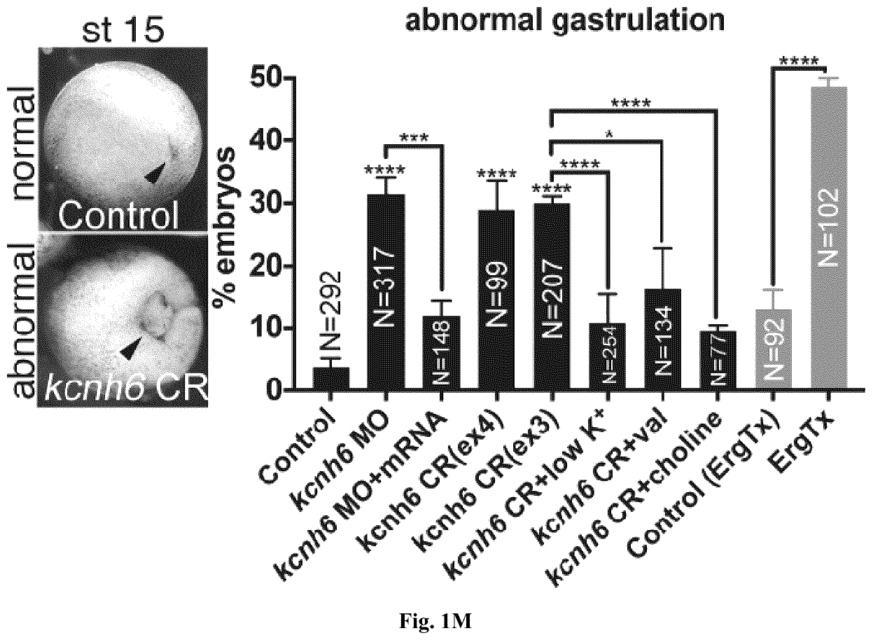

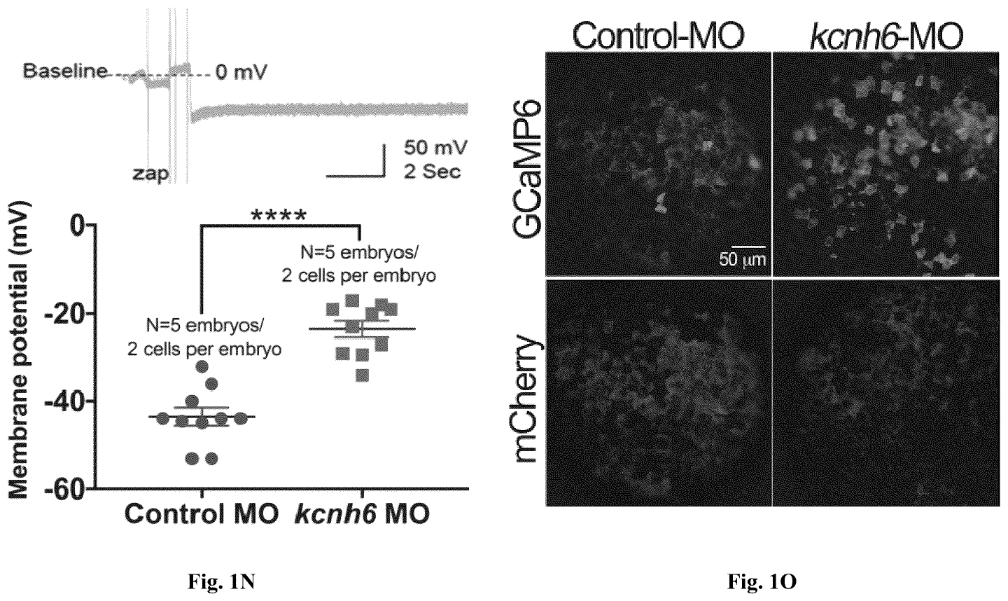

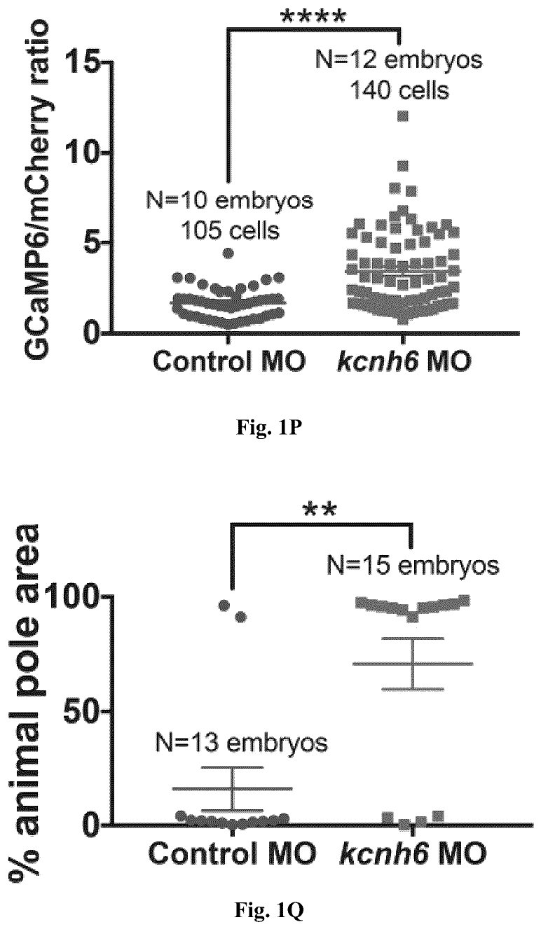

Action potentials are fundamental to the function of excitable cells, including neurons, cardiomyocytes and pancreatic cells. They are produced through tightly orchestrated changes in the membrane potential (Vₘ). However, most animal cells, excitable or not, have a resting state Vₘ (resting membrane potential) that depends on a) the permeability of the plasma membrane for each ion (p in the Goldman-Hodgkin-Katz (GHK) equation, FIG. 1A, indicating the number of active ion channels), and b) the driving force for each ion across the plasma membrane, determined by its electrochemical gradient (e.g. [K]i vs. [K]o in GHK equation). Although molecules that influence membrane potential have established roles in excitable tissues, their functions in embryonic or adult “non-excitable” tissues are emerging. For example, Vₘ appears to be crucial for the formation of the left-right (LR) body axis. While the vertebrate body plan may appear symmetrical across the LR axis, some of the internal organs, including the heart and gut, require asymmetry across the LR axis for proper formation or function. Interestingly, chemical inhibition or overexpression of ion channels or pumps disrupt the proper alignment of internal organs along the left-right axis and affect global LR patterning. Notably, using voltage sensitive dyes, Vₘ appears to vary across the developing embryo suggesting it could play instructive roles. There is now a growing field that has implicated Vₘ in various embryonic contexts including Drosophila wing patterning, craniofacial morphogenesis and chondrogenesis as well as the differentiation of excitable tissues such as muscle cells and neurons. A challenge in the field is connecting changes in Vₘ to voltage responsive effectors that lead to the gene expression changes that pattern the embryo.

In order to respond, voltage sensitive effector molecules depend on the magnitude in the change of Vₘ. Quantitative Vₘ measurements in early embryos are rare but were performed in the 1960s and 1970s from 1 cell stage embryos through blastula stages in Triturus and Xenopus embryos. The blastula embryo has completed a series of rapid cell divisions (cleavages), has established germ layer cell fates (ectoderm, mesoderm, and endoderm), and is poised to begin gastrulation, the process by which cell movements transform the embryo to acquire the adult body plan. Notably, while the Vₘ at early cleavage stages is depolarized (=more positive Vₘ) (Vₘ@2-cell=−19±10 mV), it becomes progressively hyperpolarized (=more negative) towards blastula stages (−50 mV). The implications of this progressive Vₘ polarization during early development are unclear, as is a mechanism by which Vₘ could transduce a signal within embryonic cells or act complementary to signals transduced biochemically (i.e. ligand-receptor).

### Example 2-2: V m Polarization at the Blastula/Gastrula Stage is Essential for Gastrulation and LR Patterning

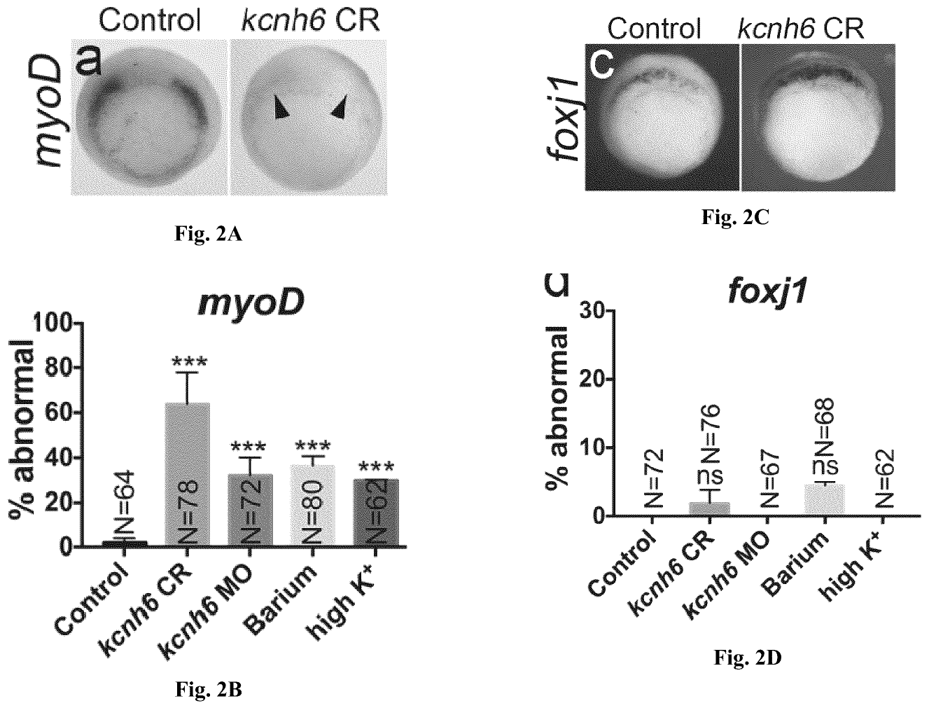

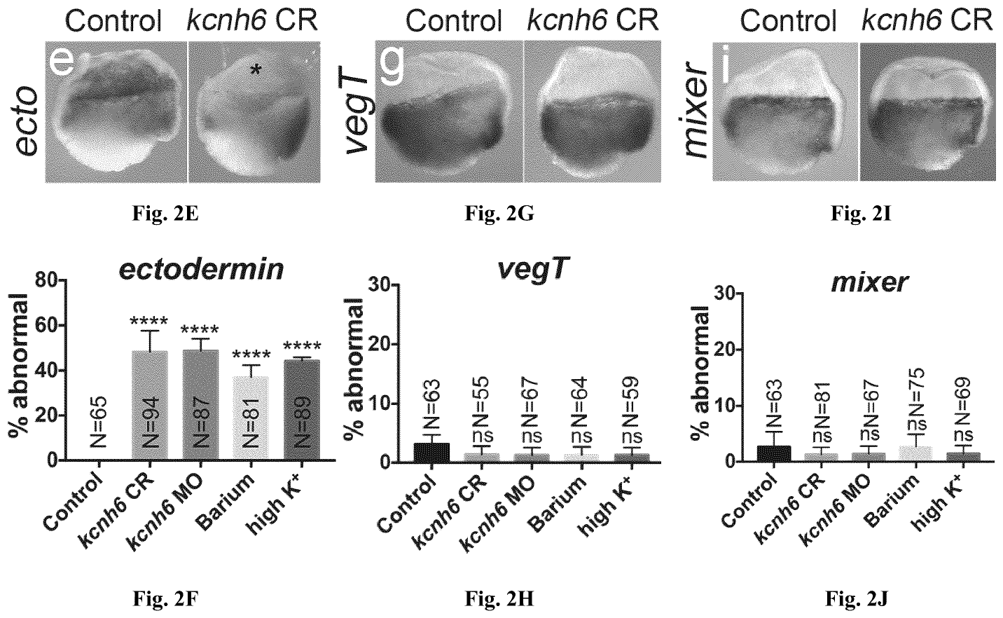

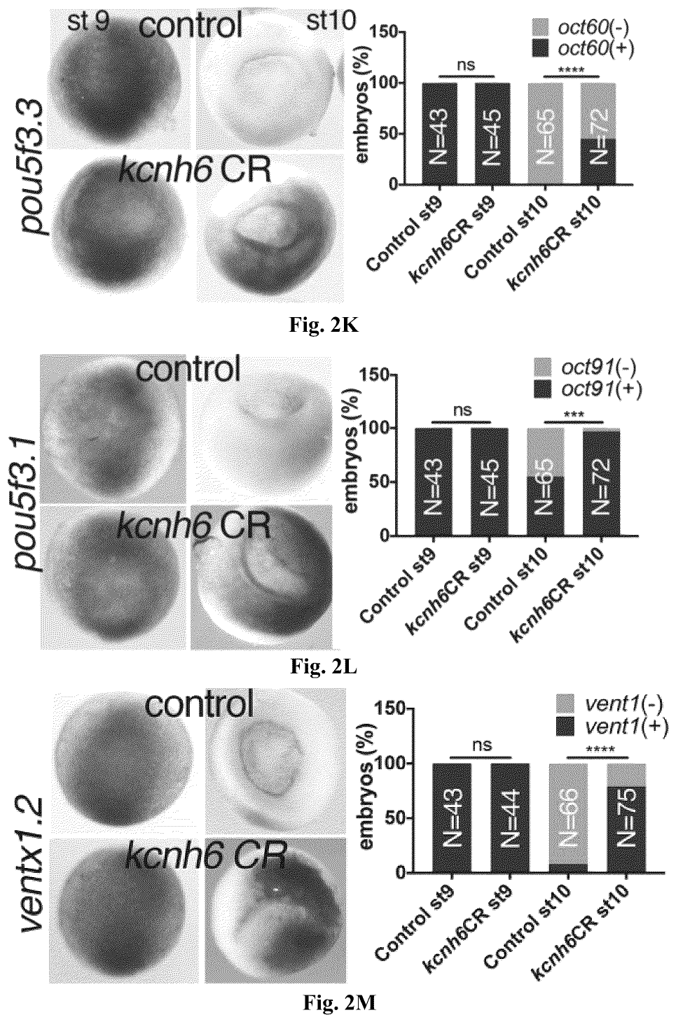

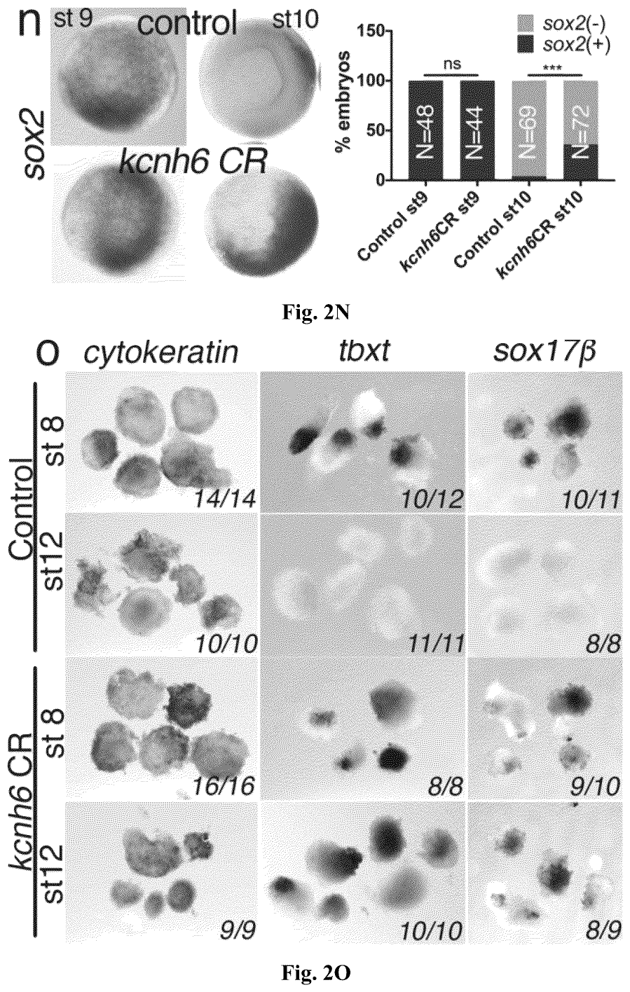

To address the question of when Vₘ is critical for embryonic development, the present study employed barium ions to block K⁺ channels at different time points of embryonic development, since K⁺ conductance is paramount for determining Vₘ. Because K⁺ conductance drives the membrane voltage to a negative (hyperpolarized) potential, blocking K⁺ channels depolarizes cells. In line with the electrophysiological evidence demonstrating that embryos first become polarized at the blastula stage, the present study found that Barium treatment affected embryonic development primarily when embryos were treated from blastula stages through gastrulation rather than at earlier cleavage stages (FIGS. 1B, 1C, and 1J-1L). Embryonic development was affected in two ways: 1) 37±3% (SEM) of the embryos failed to complete gastrulation (compared to just 3±1% in control embryos) (FIGS. 1B, 1C, 1J and 1K), and 2) 23±2% that completed gastrulation exhibited misplacement of their organs relative to the left-right axis (compared to just 4±2% in control embryos; FIGS. 1F, 1G, 1J and 1L); these included abnormal heart looping to the left, an L-loop (vs a normal D-loop to the right), inverse gut rotation and misplacement of the gall bladder on the left (vs a normal positioning of the gall bladder on the right side of the body axis) (FIGS. 1F and 1G). Because Barium can affect more than just K⁺ channels, the present study tested an alternative strategy for achieving membrane depolarization, namely manipulating Vₘ by increasing extracellular potassium ([K]o in GHK eq. FIG. 1A). Increasing the extracellular potassium reduces the chemical driving force for potassium to leave the cell and decreases the potassium current which depolarizes the embryo (more positive Vₘ). Incubating embryos in high K⁺ at blastula/gastrula stages was sufficient to cause a) gastrulation failure in 32±6% of embryos (compared to just 5±1% in controls FIGS. 1B, 1D and 1K) and b) defective organ situs at later stages in 28±3% of embryos (compared to just 4±2% in controls; FIGS. 1F, 1H and 1L). Thus, the results suggest that establishing proper Vₘ at blastula stages is essential for both gastrulation and LR patterning, providing context to work showing that Vₘ varies during embryonic development by becoming steadily more polarized from egg to blastula.

### Example 2-3: KCNH6 is Essential for LR Patterning and Gastrulation

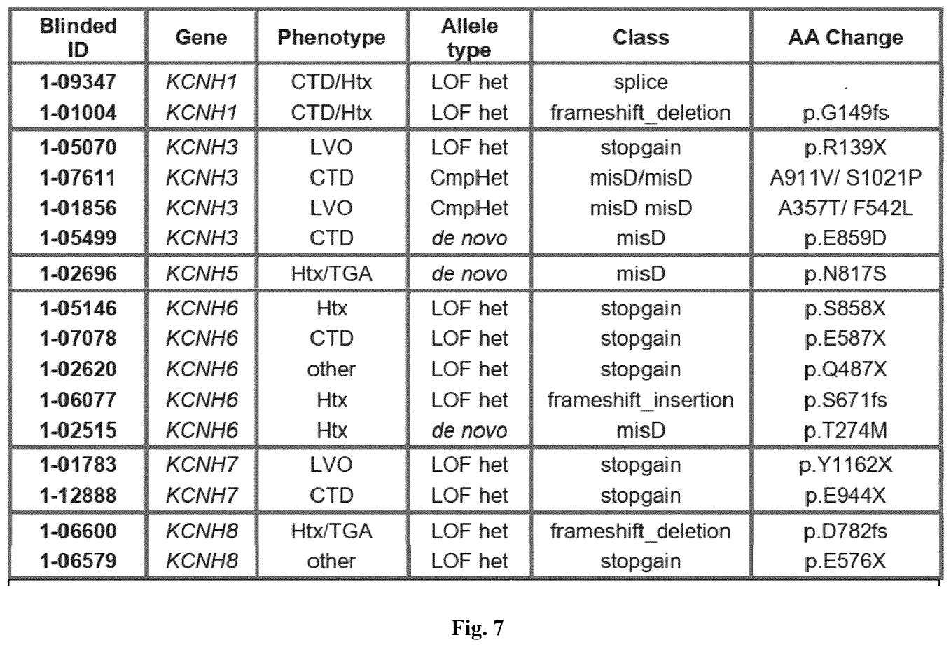

Recent studies in patients with congenital heart disease identified a number of variants in KCNH ether-a-go-go (EAG) potassium channels (FIG. 7) as candidate disease genes. Many of these patients had heterotaxy, a disorder of LR development that can have a significant impact on the structure and function of the heart that can be life-threatening. While multiple ions can affect membrane potential, the flow of potassium down its electrochemical gradient (K⁺ in>>K⁺ out) has the largest impact on Vₘ because in most cell types cell membranes are most permeable to potassium. Since KCNH6 was the most common family member in a total of five patients with heterotaxy (FIG. 7), the present study began by examining the CHD/Htx candidate gene, KCNH6.

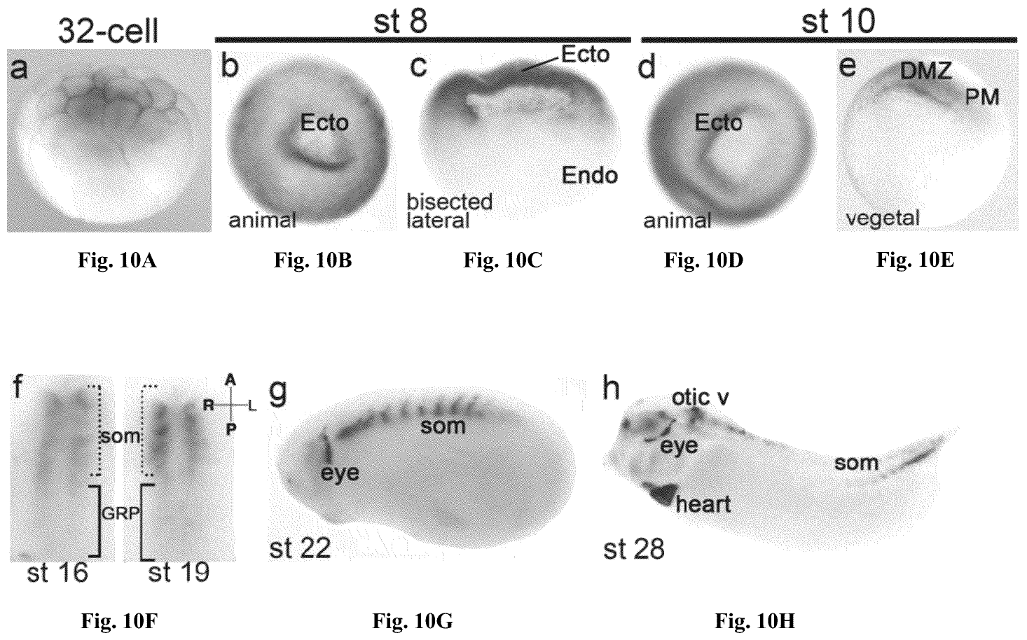

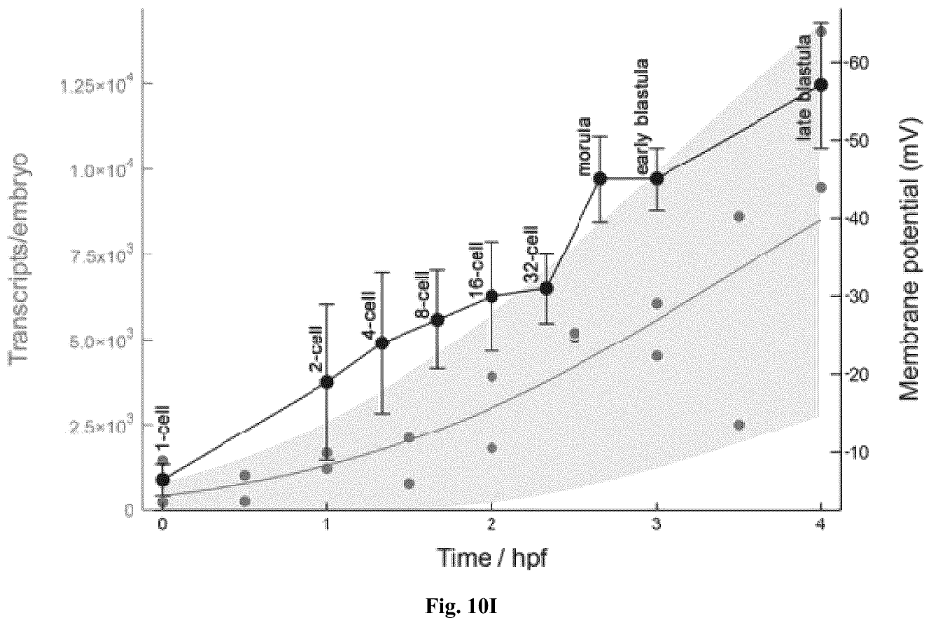

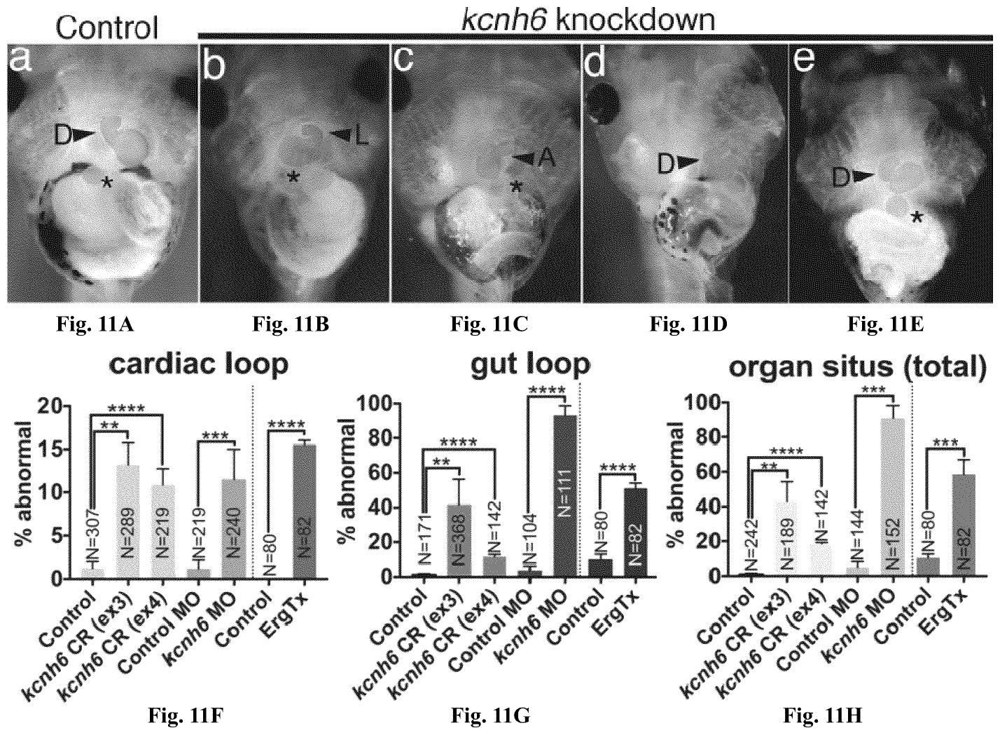

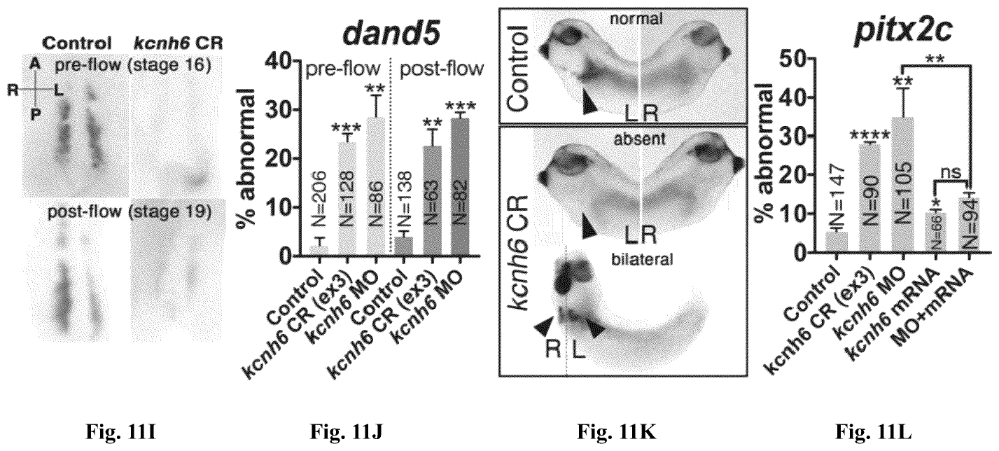

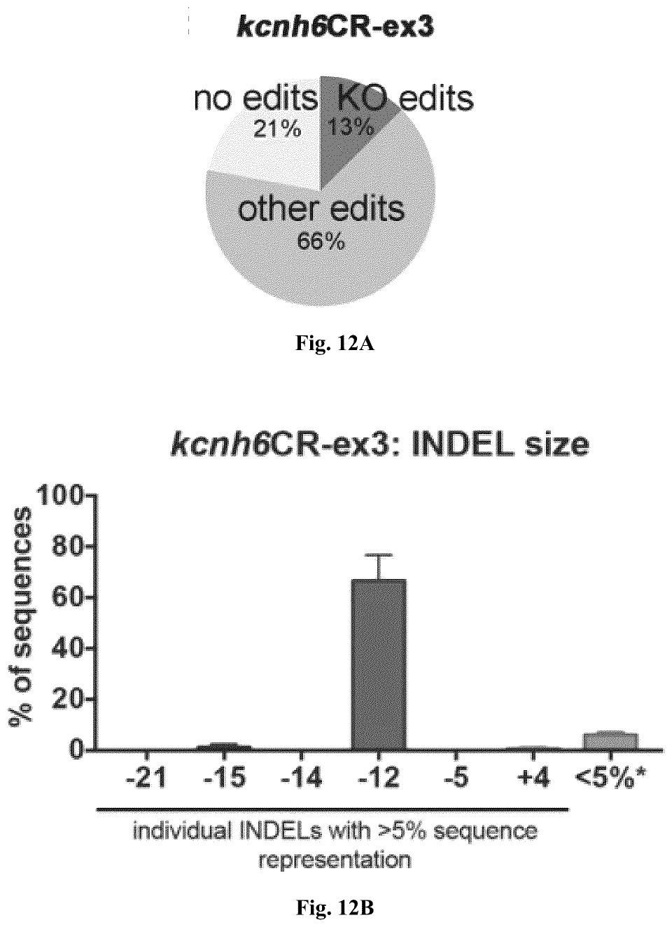

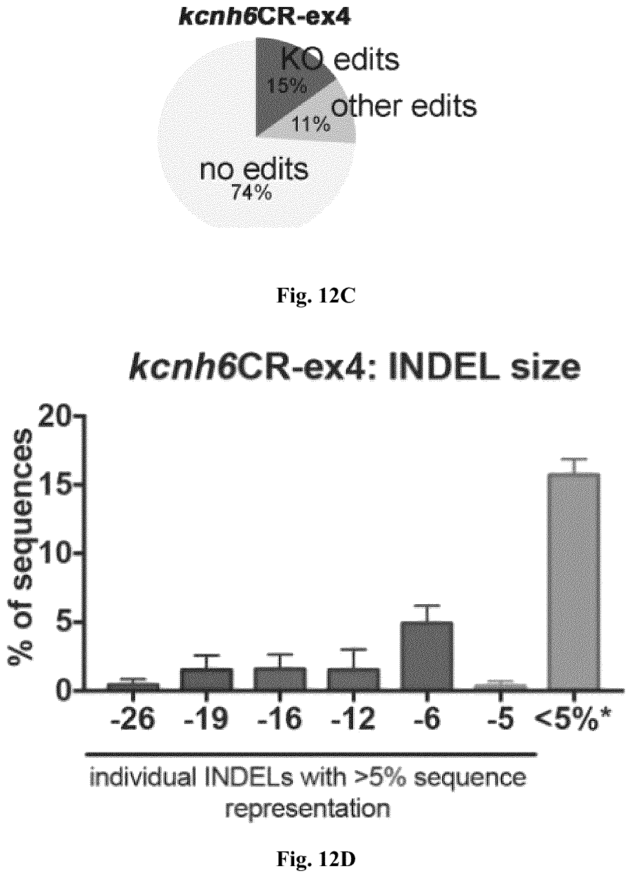

In Xenopus, the present study found kcnh6 to be expressed in the prospective ectoderm and dorsal/paraxial mesoderm at gastrulation onset, suggesting that it could play a role during gastrulation (FIGS. 10A-10H). Additionally, high temporal resolution RNA-seq shows that the increase in kcnh6 transcripts parallels the trend of Vₘ polarization in the frog embryo (FIG. 10I). The present study thus tested a role for kcnh6 in early embryonic development, and in determining Vₘ specifically. Two F0 CRISPRs independently targeting two different exons in kcnh6 as well as a translation blocking morpholino oligo (MO) recapitulated the morphological defects observed in embryos depolarized with barium and high K⁺, including: a) inability to complete gastrulation (FIGS. 1B, 1E and 1M) (CR-ex3: 27±4%; CR-ex4: 29±5% and MO:31±3% vs only 4±1% in controls) and b) abnormal organ situs 132 (FIGS. 1F, 1I and 11A-11H) (CR-ex3: 43±12%; CR-ex4: 19±1% and MO:91±7% vs only 5±3% in controls). Importantly, the present study established specificity of the depletion studies by the following criteria: 1) phenocopy with two non-overlapping sgRNAs via F0 CRISPR and one translation blocking MO (FIG. 1M), 2) rescue of the MO phenotype with human KCNH6 mRNA (FIG. 1M), and 3) detection of gene editing of the kcnh6 locus by PCR amplification and Inference of CRISPR Edits (FIGS. 12A-12D). Moreover, treatment of blastula/gastrula embryos (stage 8 to stage 12) with Ergtoxin, a scorpion peptide that specifically acts as a pore blocker of the KCNH channel family20, also led to identical gastrulation and LR defects (FIGS. 1M and 11F-11H). These results indicate that Kcnh channels, and specifically Kcnh6, contribute to gastrulation and LR development, consistent with their identification in patients with Htx/CHD.

### Example 2-4: Vₘ, Rather than KCNH6 Per Se, is Essential for Gastrulation and LR Patterning

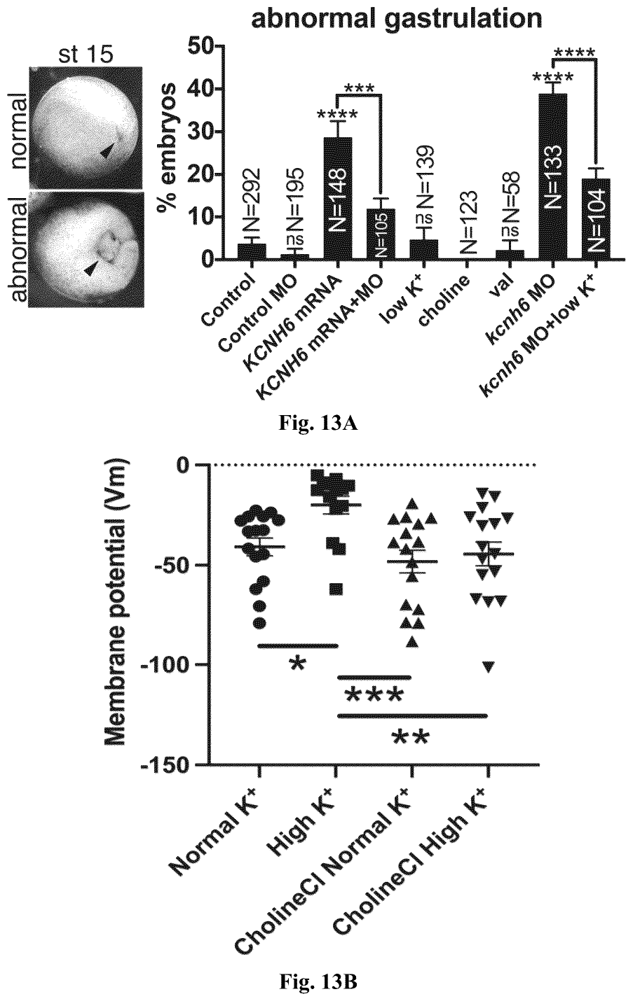

Depletion/inhibition of potassium channels or elevation of extracellular K⁺ should lead to membrane depolarization. Thus, it was reasoned that the inverse condition, namely hyperpolarizing by reducing extracellular K⁺, should rescue kcnh6 depleted embryos. Lowering extracellular K⁺ ([K]o in GHK eq. FIG. 1A) increases the outward driving force for flow of K⁺, provided that other K⁺ channels are present. Indeed, lowering extracellular K⁺ rescues the gastrulation defect in kcnh6 depleted embryos (CRISPR and MO) (FIGS. 1M and 13A-13B). To test the significance of K⁺ conductance independently of a specific K⁺ channel, the present study employed valinomycin, a K⁺ selective ionophore that inserts itself into the plasma membrane and mimics the ability of a K⁺ channel to passively conduct K⁺ down its electrochemical gradient. Application of valinomycin also rescued the gastrulation defect in kcnh6 depleted embryos (30±1% gastrulation defect in kcnh6 CR/DMSO- vs 16±7% in kcnh6 CR/Valinomycin-treated embryos) emphasizing the importance of K⁺ flux rather than a specific need for Kcnh6 itself or an alternative role of Kcnh6 in cell signaling (FIGS. 1M and 13A-13B). Finally, to differentiate between membrane potential and an alternative role for K⁺ flux across the plasma membrane (e.g. cell volume regulation), as well as to unlink membrane potential from a specific conductance (i.e. potassium), the present study hyperpolarized by reducing extracellular Na⁺ and replacing it with equimolar choline. Choline has equivalent cationic charge to Na⁺ but cannot pass through channels and therefore does not influence the Vₘ. Importantly, replacement of Na⁺ with equimolar choline does not affect the osmotic properties of the medium, ensuring that external Na⁺ depletion will only act on Vₘ and not on cell volume. To determine the amount of sodium to replace with choline, the present study measured Vₘ using intracellular electrodes in the animal pole in the context of high K⁺ and rescue by choline substitution of sodium. Embryos exposed to high K⁺ were depolarized (Vₘ=−20±4 mV) while embryos treated with high K⁺ and ½ sodium replacement with choline were repolarized to roughly the normal membrane potential (Vₘ=−41±4 mV) (FIG. 13B). Therefore, the present study tested ½ sodium replacement with choline on embryos and assayed for gastrulation which led to a remarkable rescue in kcnh6 depleted embryos (FIG. 1M). These data suggest that Vₘ, which is determined by the conductance of K⁺ through Kcnh6 and influenced by other K⁺ and Na⁺ channels, is key to gastrulation.

Finally, the present study sought to measure the change in Vₘ when kcnh6 is depleted. Using intracellular electrodes in the animal pole of kcnh6 MO vs control MO injected embryos at gastrulation onset, the present study recorded a Vₘ of −21.8±4.6 mV in kcnh6 MO vs −44±6.8 mV in control MO embryos (FIG. 1N). Thus, Kcnh6 contributes ˜20 mV to the cell's negative resting potential, and embryos lacking kcnh6 are abnormally depolarized compared to their control counterparts.

### Example 2-5: Depolarized V. Increases Calcium Levels

The present study then asked how Vₘ is transduced into a signal that affects embryonic development. There are a limited number of voltage responsive elements in a cell. It was reasoned that depolarization (Vₘ=−20 mV) in kcnh6 depleted embryos could aberrantly activate voltage-gated Ca′ channels (VGCCs), which facilitate inward Ca′ flux. L-type VGCCs are present in the prospective ectoderm and dorsal mesoderm and can induce potent intracellular Ca′ increases that can alter germ layer patterning, yet upstream regulators of these calcium channels remain elusive. Interestingly, intracellular Ca′ is elevated after fertilization and during early cleavage stages but declines as the embryo approaches gastrulation 25 concomitant with the onset of membrane polarization. It was argued that, if VGCCs are aberrantly activated due to an abnormally depolarized Vₘ, the present study should be able to detect changes in intracellular Ca′ levels. To assess this, the present study microinjected the calcium indicator GCaMP626 mRNA together with mCherry mRNA (to enable ratiometric analysis) into control MO or kcnh6 MO embryos and performed calcium imaging in animal cells of early gastrula embryos. Within the animal pole of stage 10 control MO-injected embryos, the present study observed multiple intracellular calcium increases, signified by a pulse-like appearance of GCaMP6 fluorescence in isolated cells, which then propagated to adjacent cells. These increases are well documented in Xenopus stage 8 to 12 gastrulae, i.e. last a few seconds, in which they spread to adjacent cells and then extinguish, are VGCC dependent and may contribute to neural induction. The present study confirmed the existence of Ca²⁺ transients at stage 10 by performing 20s time lapse recordings, and additionally observed that they are of low intensity and typically do not simultaneously affect more than 16±10% of the total animal pole area (FIGS. 10-1Q). Interestingly, the same transients were dramatically increased in stage 10 kcnh6 MO embryos both in intensity and area (FIGS. 10-1Q), affecting 71±11% of the animal pole on average, with most embryos displaying simultaneous calcium increases in >90% of the animal pole. Thus, kcnh6 contributes to a hyperpolarized Vₘ and is key for suppressing calcium levels at gastrulation onset, a signal that may facilitate correct gastrulation.

### Example 2-6: Depolarized Blastula Embryos Lose Paraxial Mesoderm and Ectoderm Identities

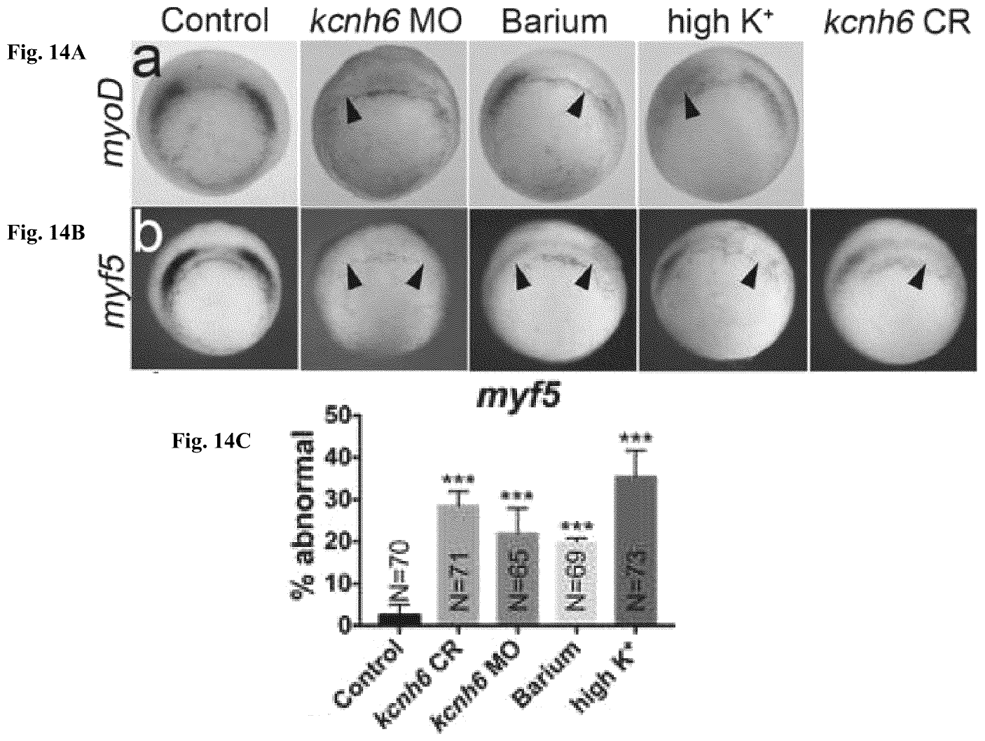

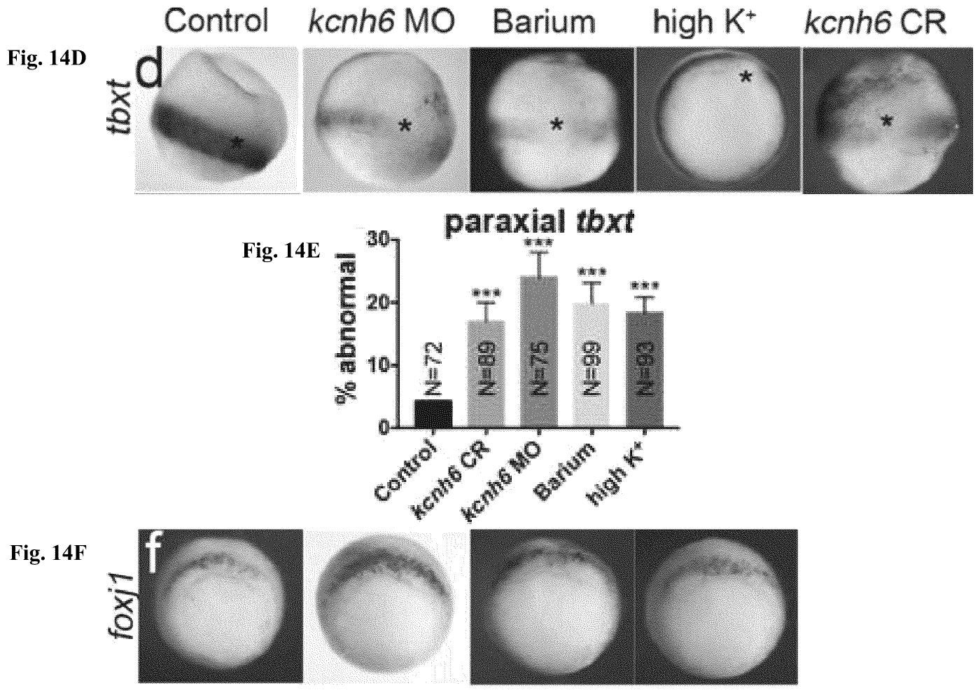

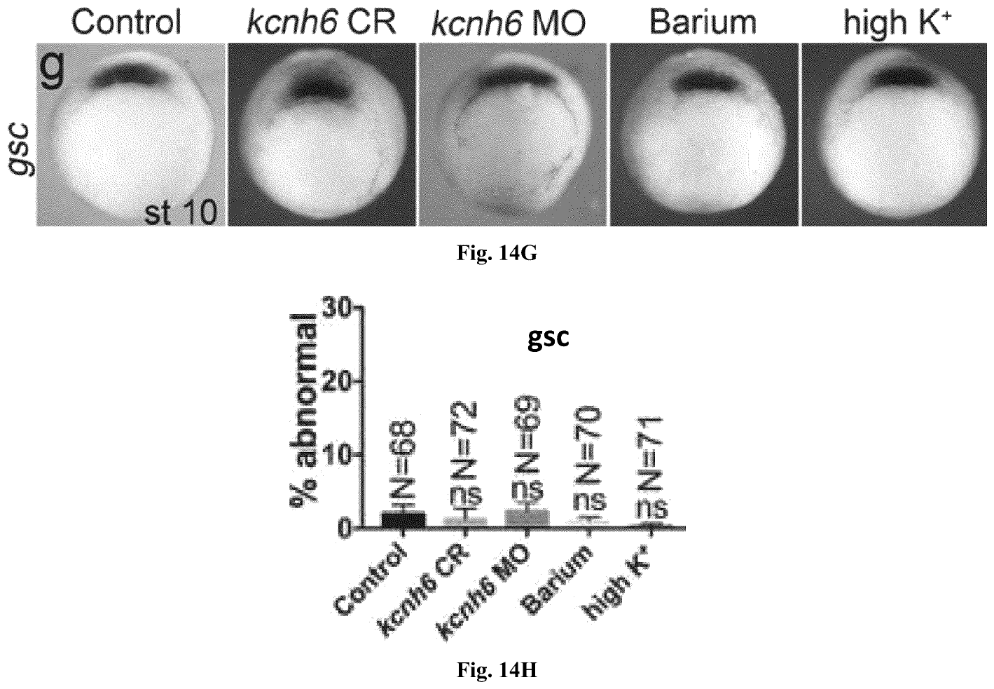

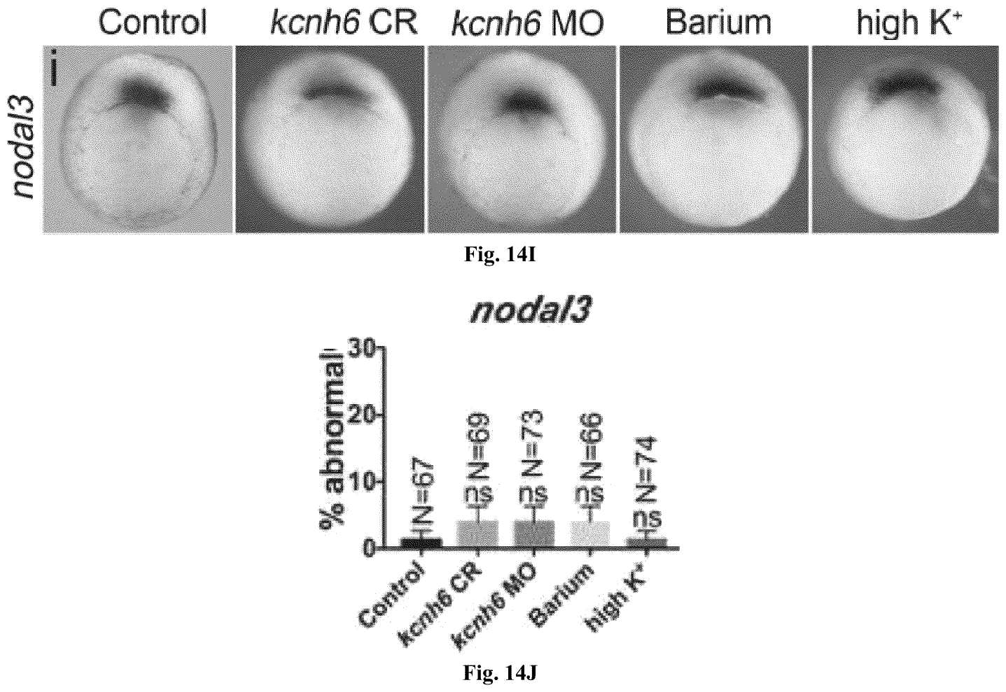

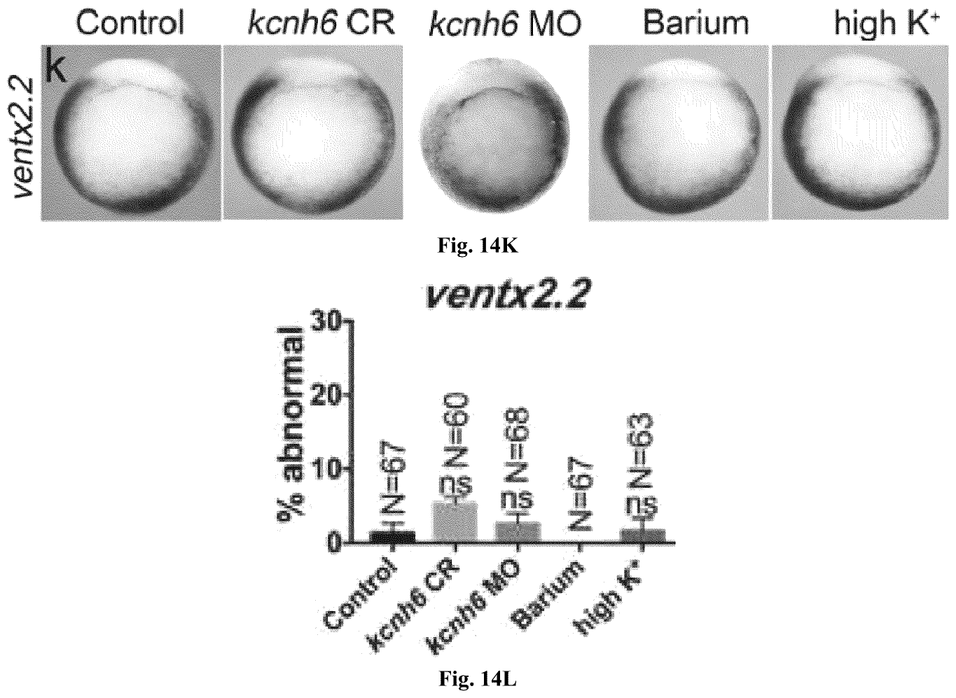

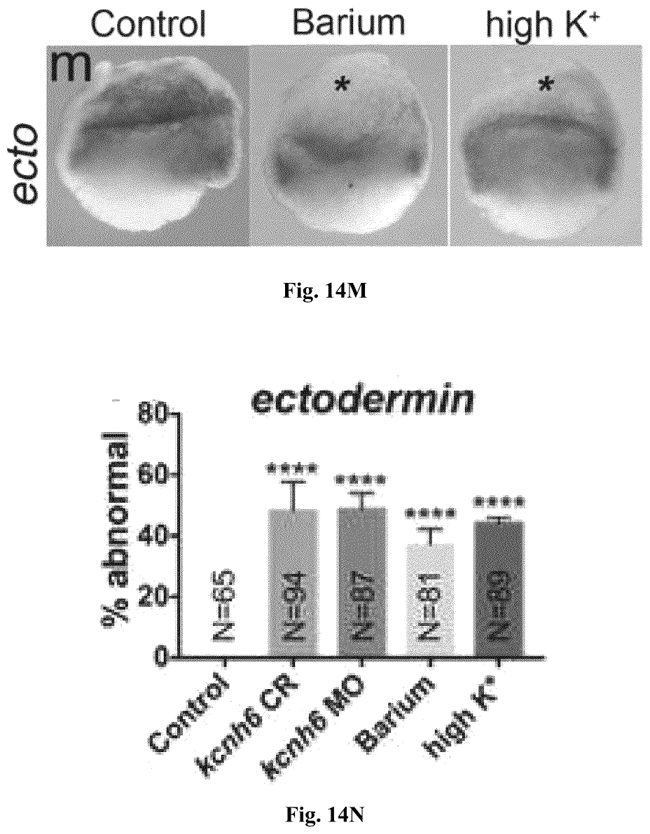

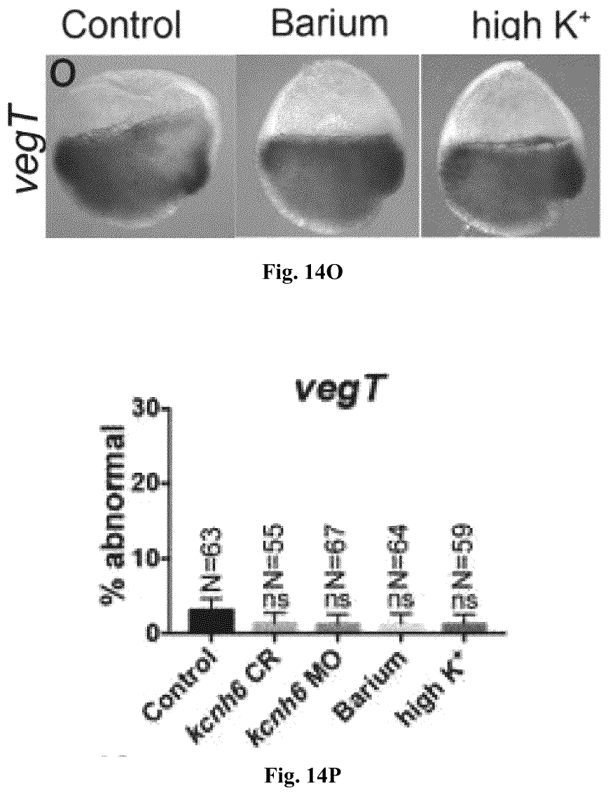

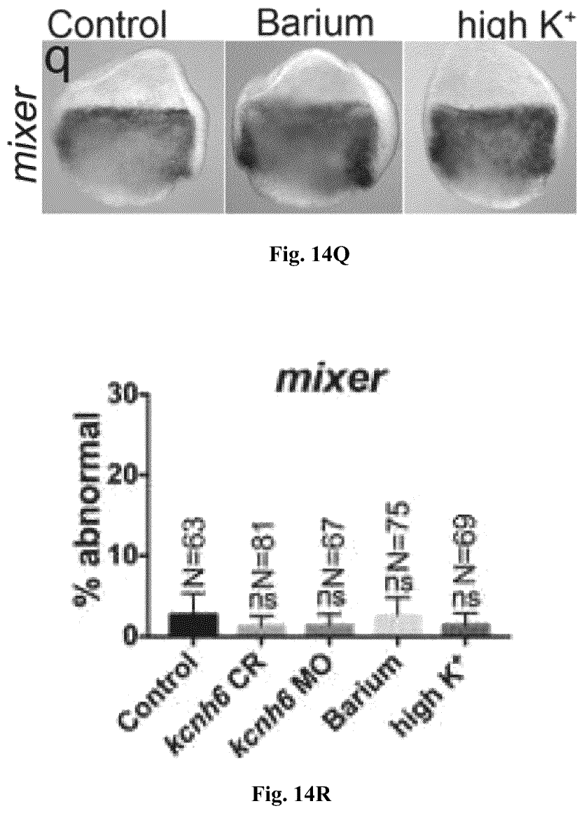

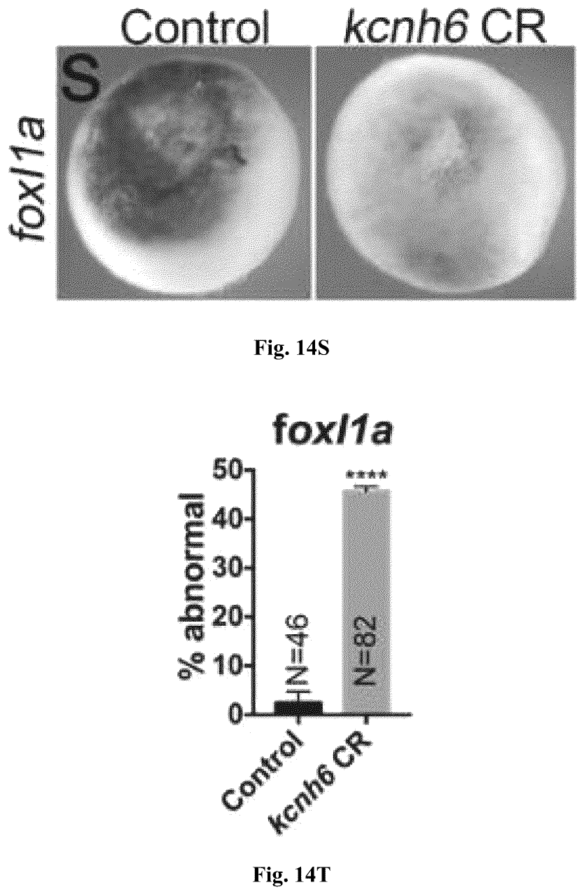

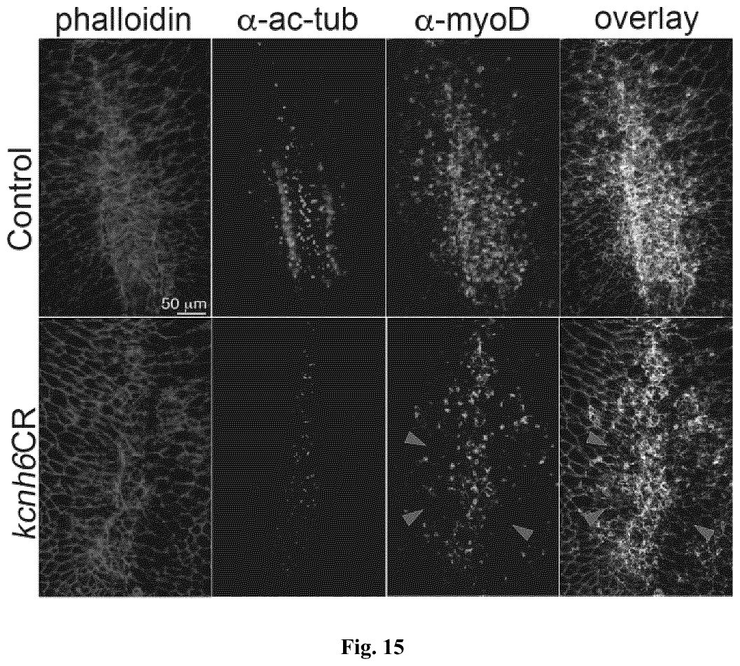

For gastrulation to proceed normally, two steps are critical: first, the germ layers of the blastula embryo (ectoderm, mesoderm, and endoderm) must be patterned correctly and second, the embryo must undergo the cellular rearrangements that drive morphogenesis. Calcium plays a role in morphogenesis cell behaviors during gastrulation. Alternatively, calcium may play a role in patterning the mesoderm that also drives gastrulation cell movements. Patterning precedes morphogenesis, and morphogenesis can fail as a result of abnormal patterning. We, therefore, first examined if patterning is disrupted in Vₘ-depolarized embryos via marker gene expression. Since the mesoderm is critical for gastrulation movements, the present study began with this germ layer. Markers of the dorsal (gsc, nodal3) and ventral mesoderm (vent2) appeared unaffected in kcnh6 depleted, barium and high K⁺ depolarized embryos (FIGS. 14G-14L); however, the paraxial mesoderm fates appeared lost as marked by myoD, myf5, and tbxt (brachyury, xbra) (FIGS. 2A-2B and 14A-14E). In fact, absent patterning of paraxial mesoderm by myoD persisted into the Left-Right Organizer (FIG. 15), a transient structure formed at the end of gastrulation where cilia driven flow is thought to pattern the LR axis. In the LRO, dand5 (coco) is normally expressed in the paraxial mesoderm symmetrically until cilia driven flow suppresses dand5 expression on the left. However, consistent with a mispatterning of the paraxial mesoderm in the LRO, dand5 was also absent even before the occurrence of cilia driven flow (FIGS. 11I-11J). A disruption in the LRO is further supported by defective pitx2c expression in the left lateral plate mesoderm at later stages (FIGS. 11K and 11L). Therefore, in kcnh6 depleted embryos, the paraxial LRO, which plays an important role in LR patterning, is mispatterned, and a defect in the patterning of this tissue can be detected already at the onset of gastrulation.

When certain biochemical signaling factors are depleted, loss of one cell fate (e.g. paraxial mesoderm) is often concomitant with gain of another cell fate. Since the dorsal or ventral mesoderm appeared unaffected (FIGS. 14G-14L), the present study considered that the ectoderm or endoderm might be expanded into the mesodermal area of embryos with abnormally depolarized Vₘ. Interestingly, while the endoderm (vegT) and its border to the mesoderm (mixer) seemed unaffected (FIG. 2G-2J), ectodermal fates (ectodermin and foxIla) were lost similar to the paraxial mesoderm (FIGS. 2E-2F and 14S-14T). In fact, depletion of ectodermin (trim33) leads to developmental arrest midway through gastrulation, which corresponds to the arrested phenotype in a portion of depolarized embryos. These results indicate that Vₘ has an effect on germ layer differentiation, and specifically paraxial mesoderm and ectoderm, at gastrulation onset (FIGS. 2A-2J).

### Example 2-7: The L-Type Voltage Gated Calcium Channel Cacna1c Responds to Vₘ Depolarization

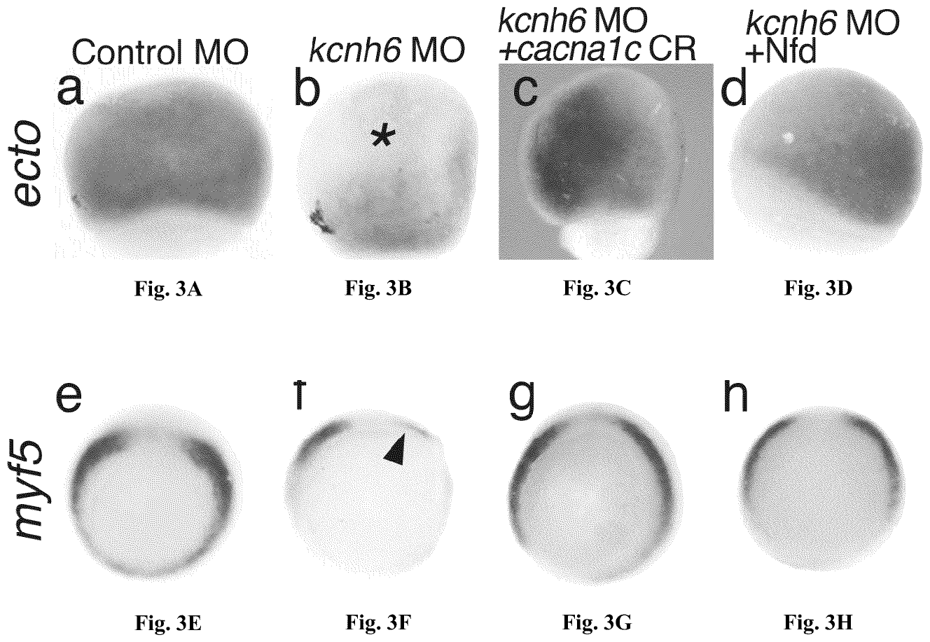

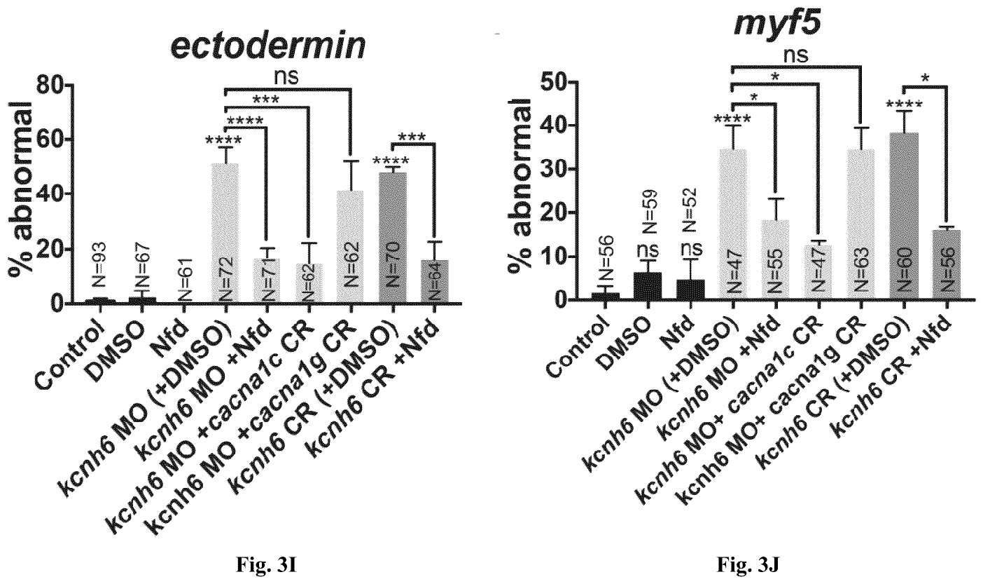

In depolarized embryos, the present study has established 1) changes in cell fate and 2) elevated intracellular calcium levels, so the present study next tested if these aberrant cell fates are dependent on voltage gated calcium channels. To determine the specific embryonic VGCCs downstream of Vₘ, the present study reviewed the available high temporal resolution RNA-Seq data. Xenopus contains detectable transcripts of L- and T-type VGCCs between the 1-cell and gastrula stages, while other VGCC types (N-, R- and P/Q) are less abundant. L-type VGCCs become activated at Vₘ>−40 mV (and then inactivated at Vₘ>10 mV) and are implicated in gastrula patterning while T-type channels become inactivated at Vₘ>−60 mV and would be inactive both at physiological Vₘ (˜−50 mV) and at more depolarized potentials. Therefore, the present study tested the L-type VGCC blocker nifedipine. This significantly ameliorated both ectodermin (ectoderm) and myf5 (paraxial mesoderm) expression losses in kcn6 knockdown embryos (FIGS. 3A-3H). Specifically, myf5 was lost only in 16±1% and 18±5% of nifedipine-treated kcnh6 CR and MO embryos (vs 38±5% and 35±5% in kcnh6CR or MO embryos treated only with DMSO, FIG. 3J). Similarly, absent ectodermin was only observed in 16±7% and 17±3% of nifedipine-treated kcnh6 CR and MO embryos (vs 48±2% and 51±6% in kcnh6 CR or MO embryos treated with DMSO, FIG. 31). Of the VGCCs identified in the RNAseq data at blastula/gastrula stages, two genes cacna1c (Cav1.2; L-type) and cacna1g (Cav3.1; T-type) encode alpha (pore-forming) channel subunits, which are indispensable for channel function. Co-depletion of cacna1c in kcnh6 depleted embryos rescued expression of ectodermin and myf5, while co-depleting cacna1g (FIGS. 3A-3J) resulted in no rescue. Thus, Kcnh6, which sets a negative Vₘ, is essential to limit the activation of L-type VGCCs and specifically Cacna1c, a critical step for ectodermal and paraxial mesodermal differentiation.

### Example 2-8: Vₘ Depolarization Maintains Pluripotency in Gastrula Embryos

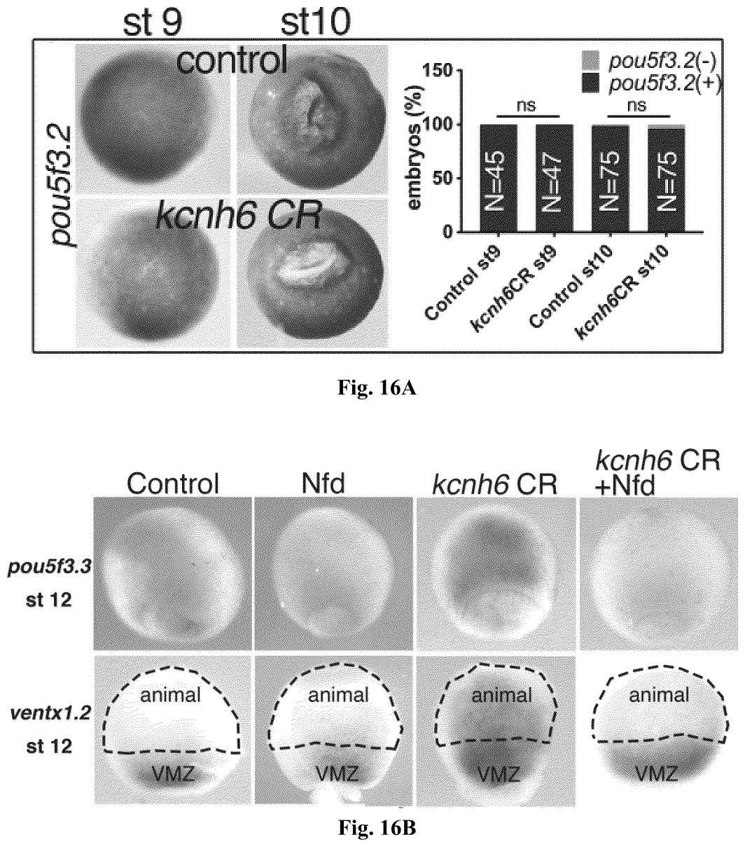

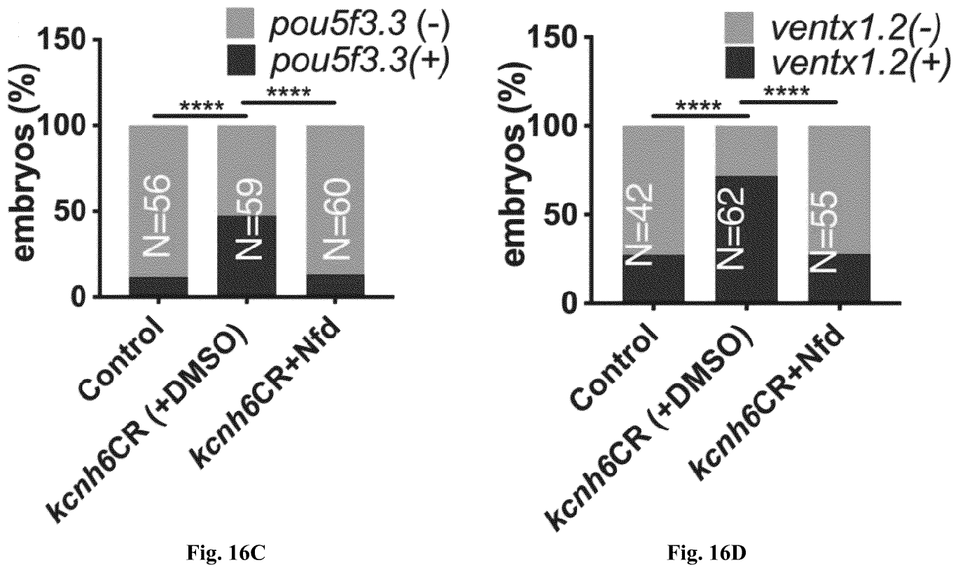

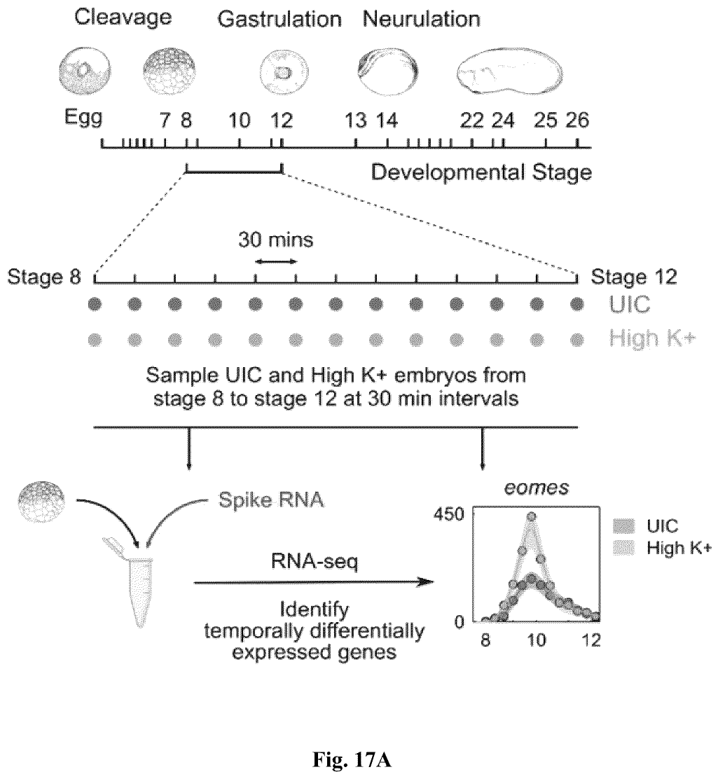

The loss of some cell fates (ectoderm and paraxial mesoderm) without a concomitant expansion of other cell fates was puzzling given that most biochemical signaling factors (Wnt, BMP, Nodal) generally balance different cell fates in the early embryo. It was speculated that these unspecified cells may simply lack the ability to assume any cell fate because they remain pluripotent abnormally. To test this hypothesis, the present study examined markers of pluripotency OCT4, NANOG, and SOX2. In Xenopus, there are three OCT4 homologs (pou5f3.1, 2 and 3, formerly oct91, oct25 and oct60), and the ventx1.2/2.2 factors, which have overlapping functions in maintaining differentiation competence and are thought to be structurally and functionally equivalent to mammalian Nanog40. Sox2, a core pluripotency factor in mammals, is highly conserved in amphibians and also expressed at high levels prior to lineage commitment throughout the Xenopus blastula41,42. The present study examined the prospective ectoderm of embryos, which is best characterized in its pluripotent properties and confirmed that pou5f3.1, pou5f3.3, sox2 and ventx1.2 are robustly expressed at stage 9 prior to lineage commitment, but their transcripts are sharply reduced by stage 10 in control embryos (FIGS. 2K-2N). In contrast, kcnh6 CR embryos retain robust expression of these factors well beyond stage 9 and into stage 10, a prolonged expression compared to wildtype embryos (FIGS. 2K-2N). This, in turn, is not due to a general delay in development, since kcnh6 CR embryos were staged according to the physical progression of gastrulation, i.e. presence of blastopore lip. Moreover, in kcnh6 depleted late gastrula embryos, abnormal maintenance of pou5f3.3 and ventx1.2 can be abolished by incubating the embryos in L-type VGCC blocker nifedipine (FIGS. 16B-16D). These results suggest that kcnh6 is upstream of Vₘ and VGCCs in promoting the exit from pluripotency, which takes place as gastrulation proceeds.

Based on this result, the present study sought to test the pluripotency of these kcnh6-depleted embryos. In the blastula (stage 9), the prospective ectoderm or “animal cap” contains cells that when explanted will differentiate into epidermis (FIG. 20). Importantly, when stage 8-9 explanted animal cap cells are treated with activin, they can be differentiated into mesodermal and endodermal cell fates indicating that they are pluripotent (FIG. 20), an assay used for decades to test the activity of a host of differentiation factors. However, towards the end of gastrulation at stage 12, these animal cap cells are no longer pluripotent and when explanted will only differentiate into epidermis, even when stimulated with activin (FIG. 20). Using this animal cap assay, the present study sought to test the role of kcnh6 in determining pluripotency. The present study explanted stage 9 and stage 12 animal caps and assayed differentiation under three conditions: 1) no activin to examine spontaneous differentiation into epidermis (marked by cytokeratin), 2) low activin to stimulate differentiation into mesoderm (tbxt) and 3) high activin to stimulate differentiation into endoderm (sox17β). Both control and kcnh6 CR animal caps explanted from stage 9 embryos were capable of differentiating into all three germ layers, indicating full differentiation potential even when kcnh6 is depleted (FIG. 2O). On the other hand, as expected, animal caps explanted from stage 12 control embryos differentiated into epidermal fate but not into meso- or endoderm despite activin administration (FIG. 2O). Strikingly, stage 12 animal caps explanted concurrently from kcnh6 depleted embryos were able to differentiate into cell fates of all three germ layers with activin administration (FIG. 2O). From these experiments, it was concluded that Vₘ polarization via kcnh6 enables the exit from pluripotency.

### Example 2-9: V. Polarization Limits mTOR Signaling to Allow for Exit from Pluripotency

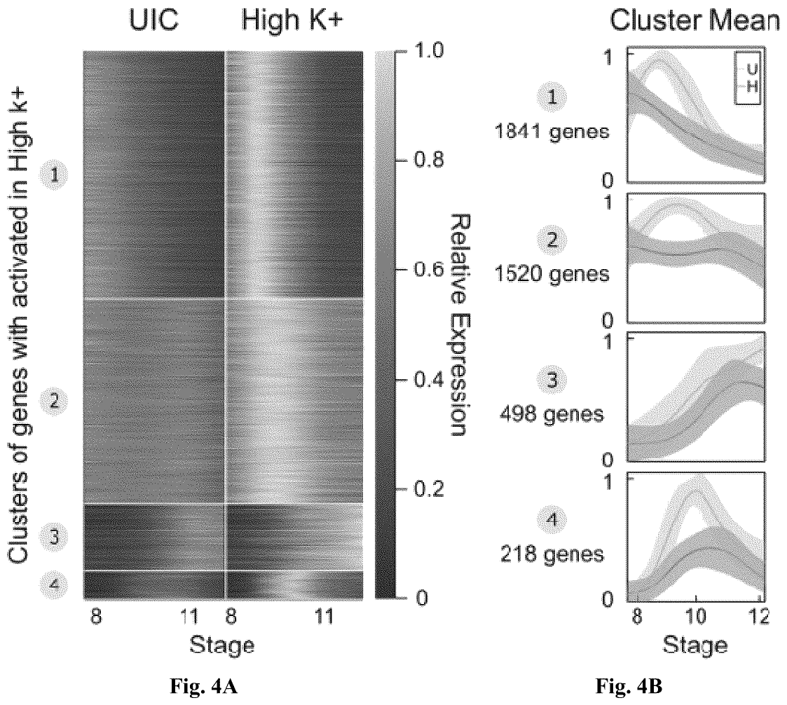

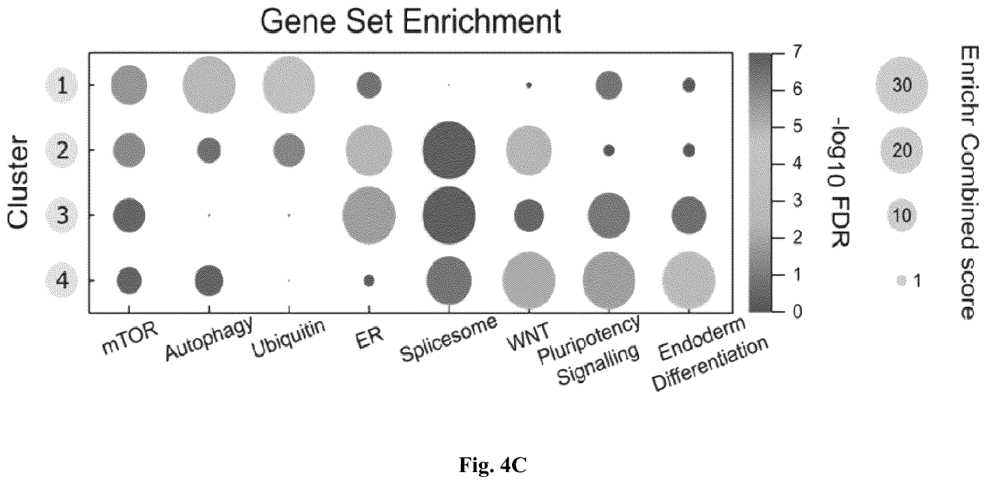

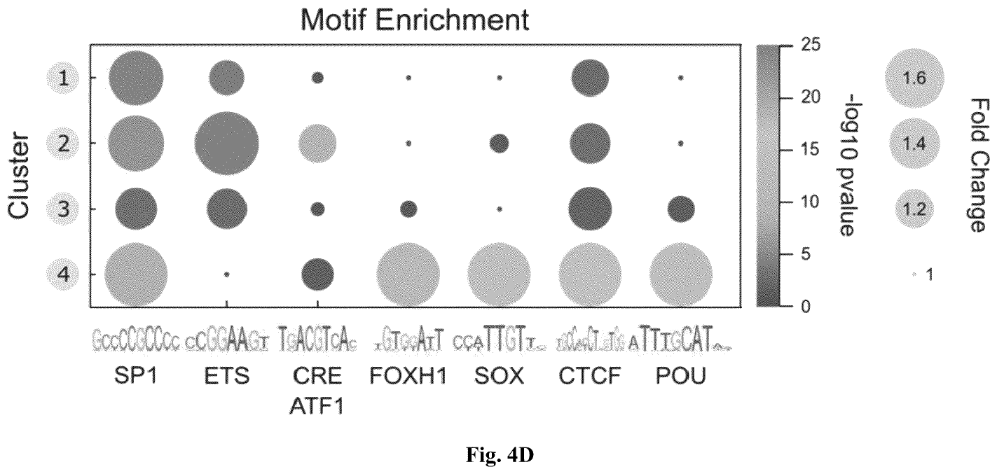

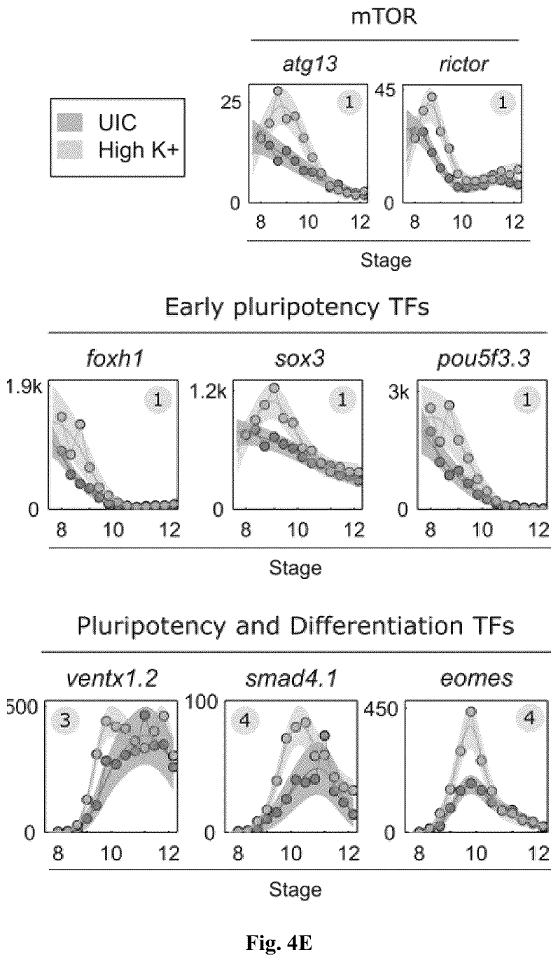

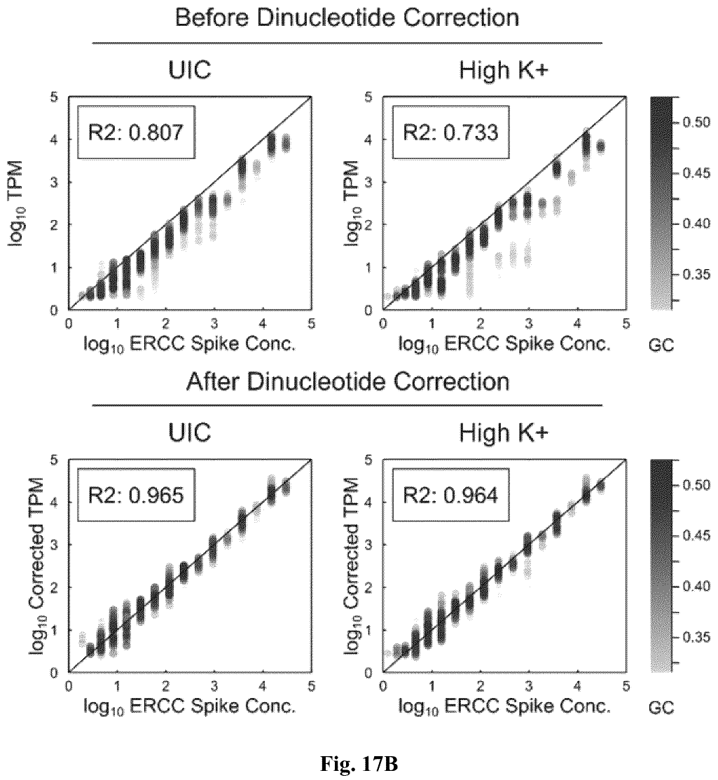

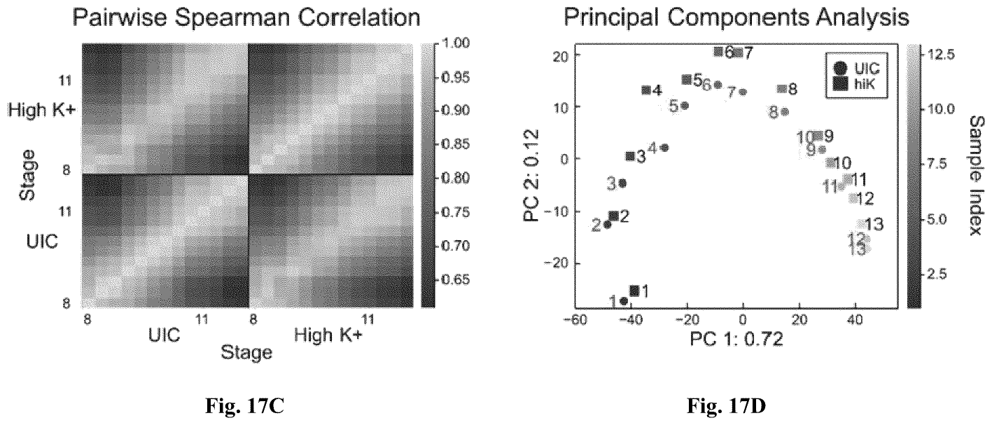

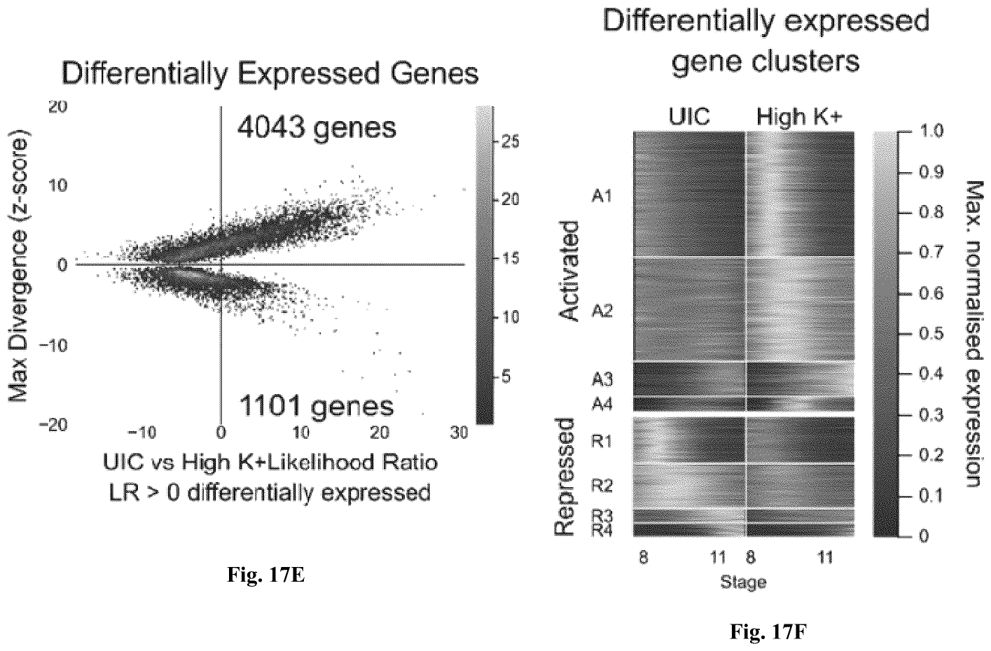

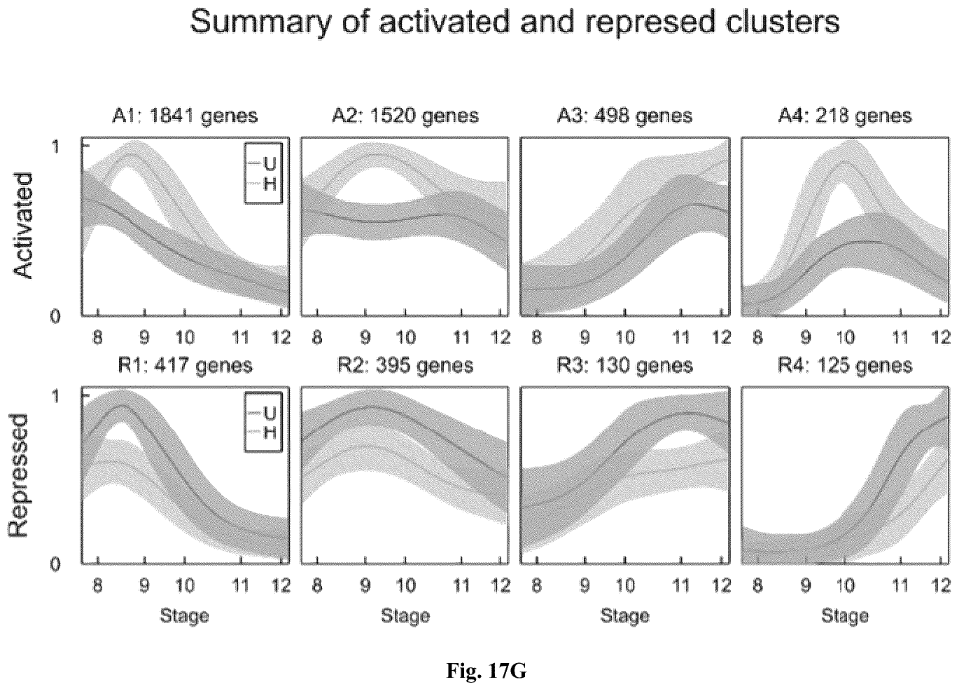

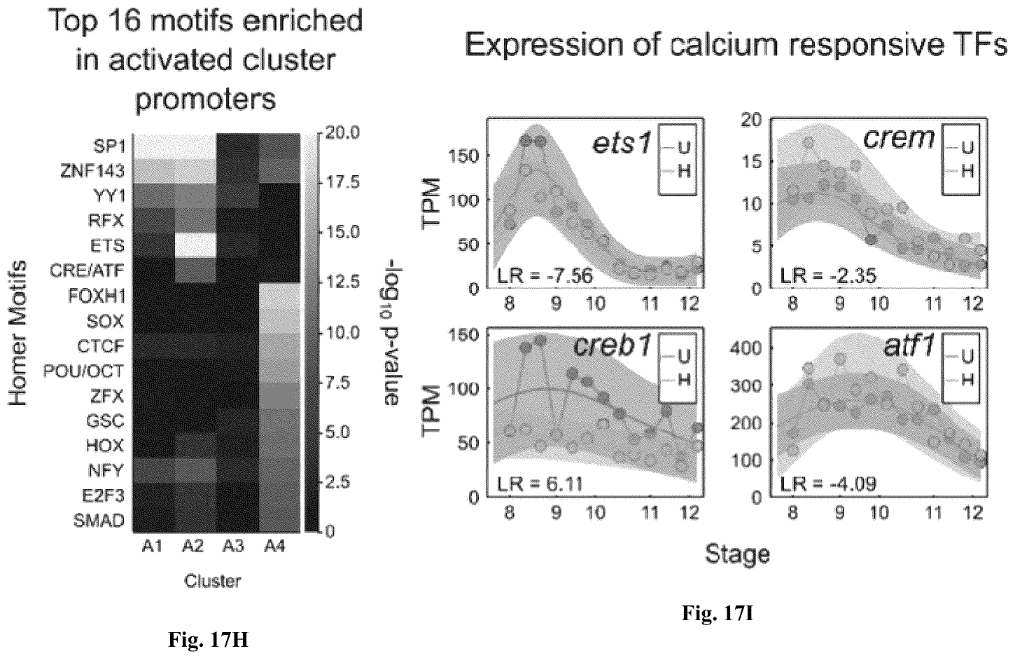

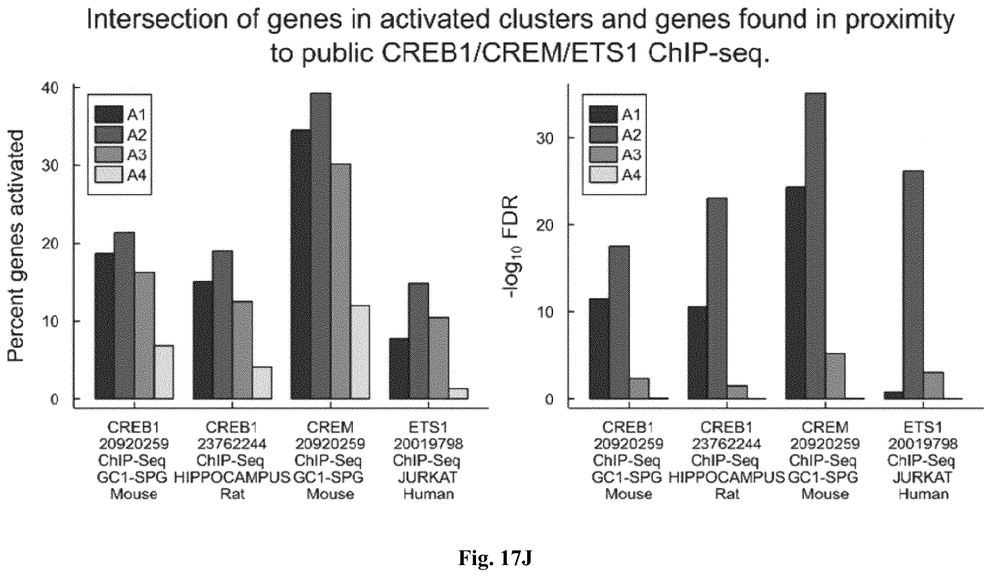

Our findings indicate that a polarized Vₘ limits voltage-gated calcium channels and intracellular calcium, a process that reduces the expression of pluripotency genes as germ layer differentiation initiates. A critical question is what are the signaling pathways invoked when Vₘ is depolarized or intracellular calcium is elevated. To address this question in an unbiased manner, the present study temporally profiled gene expression via RNA-Seq in control and high K⁺ depolarized embryos by collecting embryos every 30 min from pre- to post-gastrula stages (stages 8 to 12; FIGS. 17A-17D). The present study identified genes exhibiting temporal differential expression employing a Gaussian Process framework; this determines genes whose expression trajectory differs between control and high K⁺ embryos over the time course. The present study found 4043 genes on average activated in high K⁺ over the time course, and 1101 genes on average repressed (FIG. 17E). The present study further used k-means clustering to subdivide these into 8 clusters, 4 activated (A1-4, FIGS. 4A and 4B) and 4 repressed (R1-4, FIGS. 17F and 17G). In both cases, the clustering segregated genes showing dysregulated gene expression prior to gastrulation (Clusters 1, 2) and during gastrulation (Clusters 3,4) (FIGS. 4A, 17F and 17G). To assess the composition of these clusters, the present study performed gene set enrichment using Enrichr. Comparing activated clusters to repressed clusters over seven different annotated gene set libraries, the present study found 1279 terms significantly associated with at least one of the 4 activated clusters, but only 44 terms significantly associated with repressed clusters. Therefore, given the total number of genes and associated terms, the present study focused the attention on the analysis of the activated genes.

The present study found a hierarchy of gene set enrichments from early to late in the time course, reflecting the changing response in the transcriptome (FIG. 4C). Notably, the present study found enrichment for the mTOR signaling pathway activated early with members including rictor, depdc5, pik3cb, stk11, atg13 in cluster A1 (FIG. 4E) and akt1s1, gsk3b, lamtor1/2 in cluster A2. The enrichment for mTOR members continues to span all activated clusters (FDR<3×10⁻⁵ overall activated clusters combined). This enrichment is accompanied by pathways associated with mTOR, including autophagy, ubiquitin transferase activity, and ER response (FIG. 4C). The intermediate clusters A2 and A3 show the most prominent ER response enrichment together with significant upregulation of spliceosome machinery (including 12 snRNPs and 5 SRSF family members). In contrast, in later clusters, the present study found enrichments for terms explaining the sustained pluripotency and germ layer defects, including pluripotency (pou5f3.1, pou5f3.2, ventx1.2) and WNT signaling (fzd7, wnt8a, tcf711) (FIGS. 4C and 4E). Key members of the Xenopus pluripotency network are also found activated early, in cluster A1 including foxh1, sox3 and pou5f3.3 (FIGS. 4C and 4E).

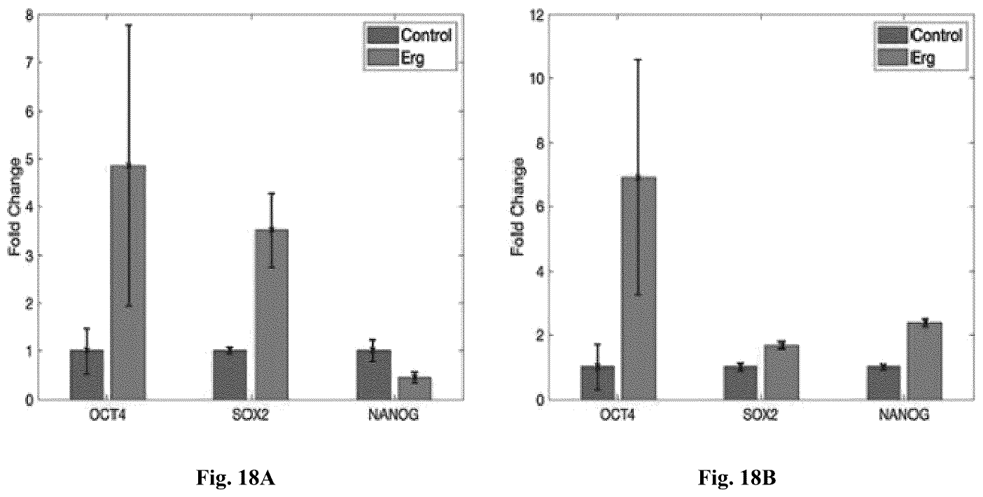

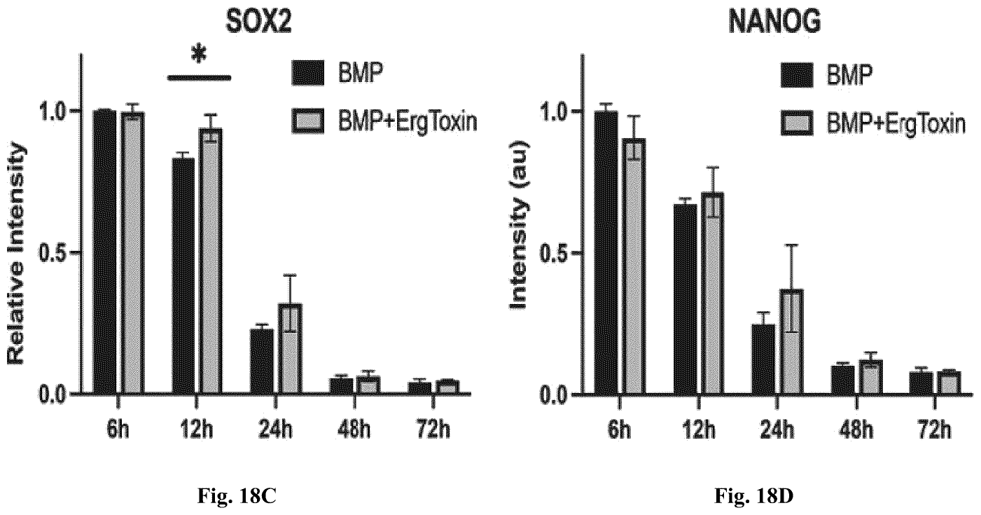

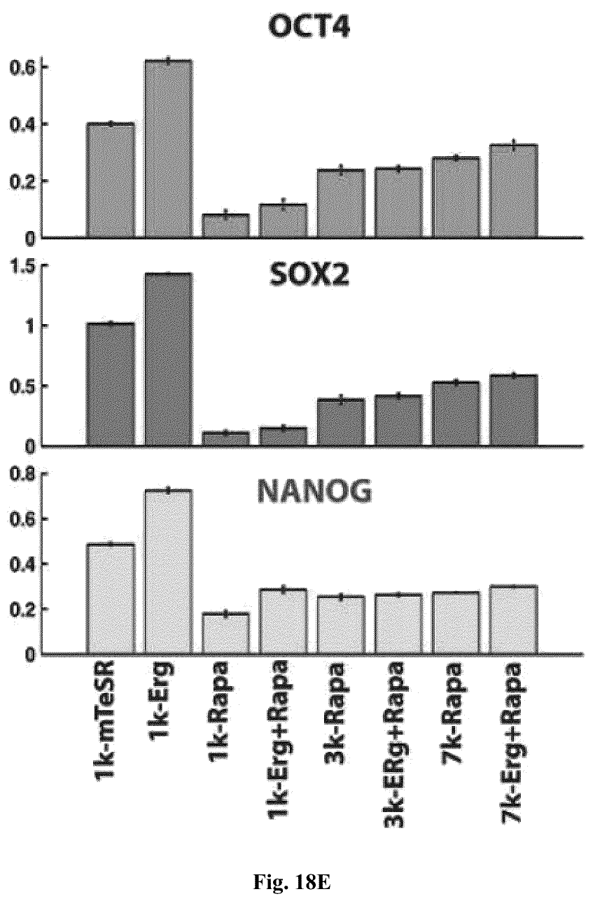

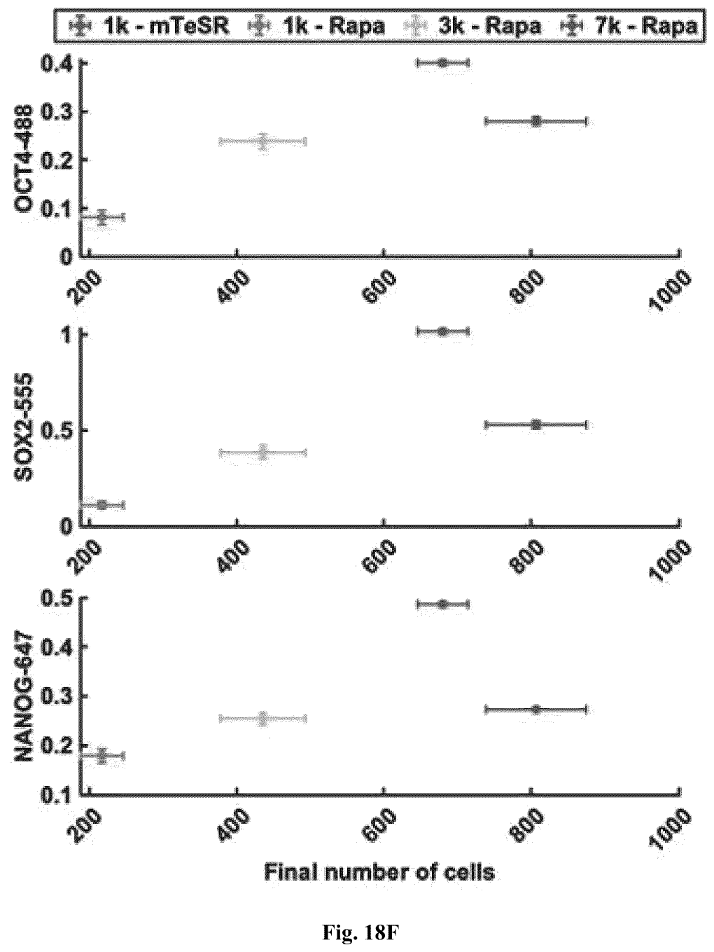

To build the underlying gene regulatory networks, the present study examined transcription factor motif enrichment in the promoters of each of these activated gene clusters. Mirroring the gene set enrichments, the present study found motif enrichments segregated between early (A1,2) and later (A3,4) gene clusters (FIGS. 4D and 17H). The present study found strong enrichment for SP1, ETS, YY1, RFX and CRE/ATF1 motifs driving gene expression changes in clusters A1 and A2. Interestingly, both ETS and CRE motifs are bound by factors responsive to calcium. Calcium induced phosphorylation of ETS1 inhibits binding activity45, and CRE elements are bound by calcium responsive family members, CREB1, CREM and ATF146-48. In the data herein, each of these factors are expressed at high levels at stage 8, and then are gradually downregulated to a minimum at stage 12. Of note, their mRNAs are not upregulated in high K⁺ conditions (FIG. 18I). This suggests that the activity of these factors is post-translationally modified in depolarized embryos experiencing high Ca²⁺ to drive gene expression changes. In the case of ETS1, this factor may act to repress gene expression in normal germ layer resolution and this repression is removed in high Ca²⁺ embryos. Supportive of these factors driving gene expression, the present study found a large intersection between activated gene clusters A1 and A2 and genes found in proximity to publicly available ETS1, CREB1 and CREM binding sites (FIG. 17J); in the case of activated cluster A2 this remarkable enrichment accounts for 794/1520 (52.2%) of genes found in proximity to one of ETS1, CREB1 and CREM (FDR<10-26,10-23, 10-35 respectively, Fisher Exact Test). Therefore, the present study found that initial transcriptional responses captured by activated gene clusters A1 and A2 appear to be largely driven directly by calcium response. This includes the activation of mTOR and pluripotency genes in high K⁺ conditions, mTOR genes show an enrichment in CRE sites in their promotors across all clusters (p≤0.0072, Odds Ratio 2.46, Fisher's Exact Test) and pluripotency genes show enrichment for CRE sites in cluster A1 and A2 (p≤0.017, Odds Ratio 3.08, Fisher's Exact Test) and ETS sites in cluster A2 (p≤0.021, Odds Ratio 5.15, Fisher's Exact Test).

Turning to the genes activated later, particularly, those associated with pluripotency and germ layer commitment in cluster A4, the present study found comprehensive enrichment of FOXH1, SOX and POU motifs in their promoters (FIG. 4D). These motifs correspond precisely with the early pluripotency TFs whose transcripts are activated in cluster A1 (FIG. 4E). Together, the high-resolution temporal profiling of the transcriptome in control and high K⁺ conditions reveals a cascade of transcriptional activation. The present study proposes a model where a depolarized membrane opens VGCCs and elevates intracellular calcium leading to the expression of transcripts (including mTOR and pluripotency factors) whose promoters are enriched with calcium responsive motifs. This is followed by the activation of transcripts involved in pluripotency and germ-layer commitment, driven by the pluripotency factors activated in the early wave of gene expression.

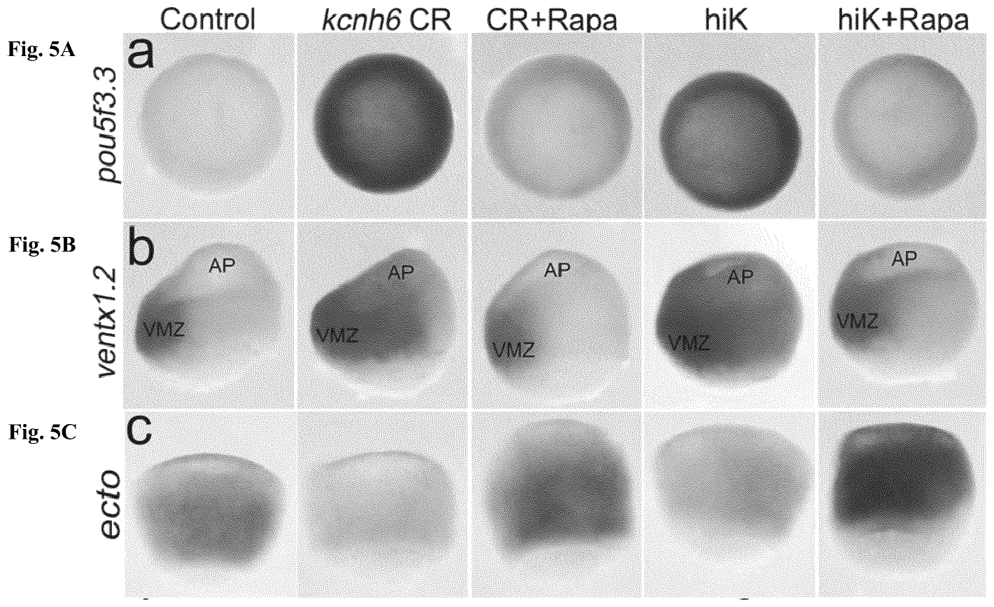

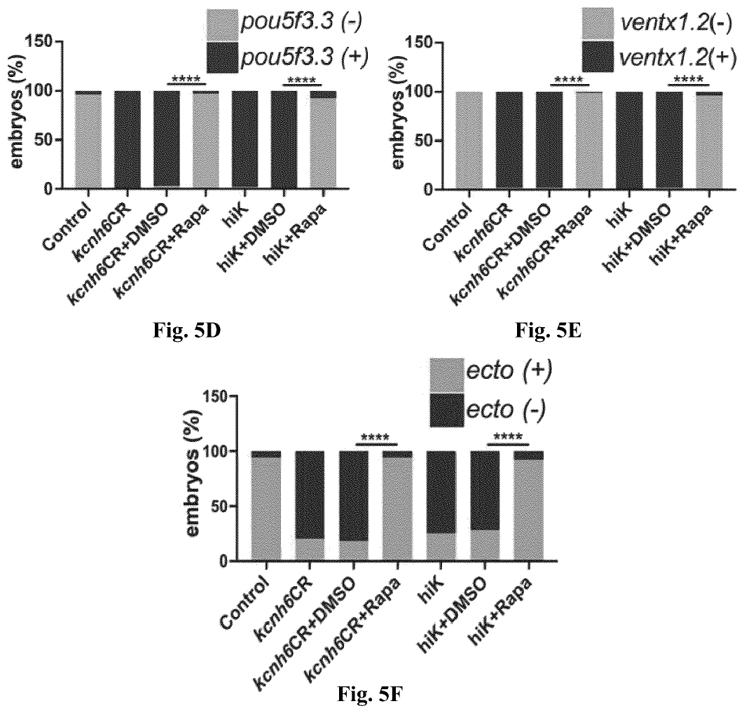

The transcriptome analysis herein not only revealed a potential gene regulatory network but pointed towards a role for mTOR. mTOR is critical for multiple cellular processes including autophagy, nutrient sensing, and an emerging role in pluripotency. Because the expression of mTOR pathway members was increased in depolarizing conditions and pathways associated with mTOR, it was reasoned that mTOR signaling was upregulated and maintained pluripotency in these depolarized embryos. To test this hypothesis, the present study applied the mTORC1 inhibitor, rapamycin, to depolarized gastrulating embryos to see if this could abolish the aberrant expression of pluripotency markers pou5f3.3 and ventx1.2 in the animal pole and activate germ layer differentiation. Rapamycin dramatically lowered expression of pou5f3.3 and ventx1.2 in the animal pole in kcnh6 CR and high K⁺ treated embryos compared to those embryos treated with vehicle alone and appeared comparable to untreated control embryos (FIGS. 5A, 5B, 5D and 5E). Conversely, the expression of the ectodermal marker, ectodermin, which was reduced in depolarizing conditions (kcnh6 depletion or exposure to high K⁺), was recovered with rapamycin treatment (FIGS. 5C and 5F). Therefore, a polarized Vₘ at gastrulation onset is critical for limiting mTOR in order to suppress pluripotentcy genes and enter differentiation.

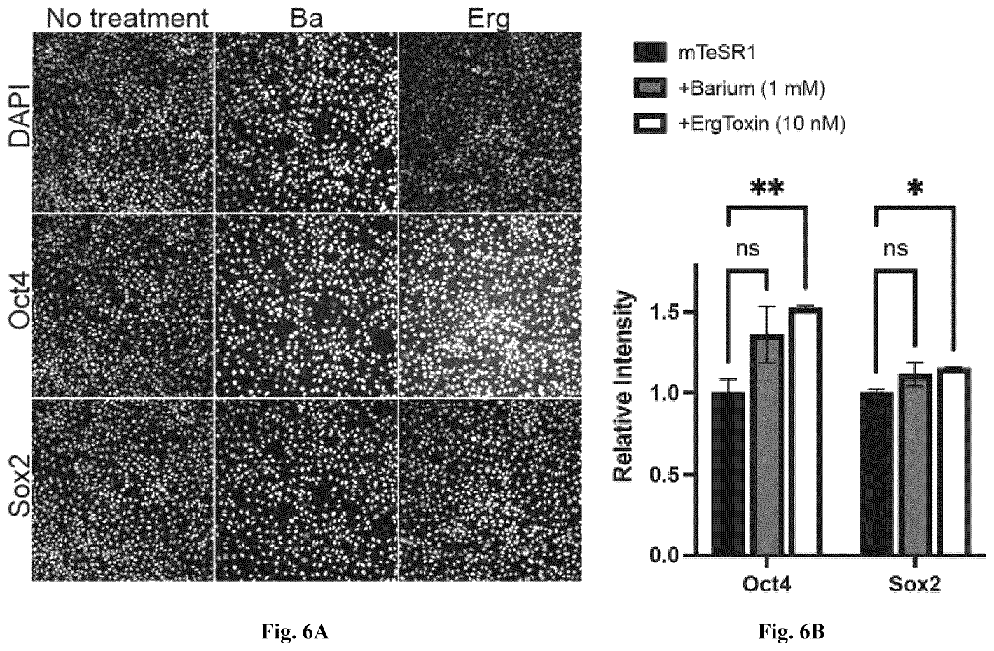

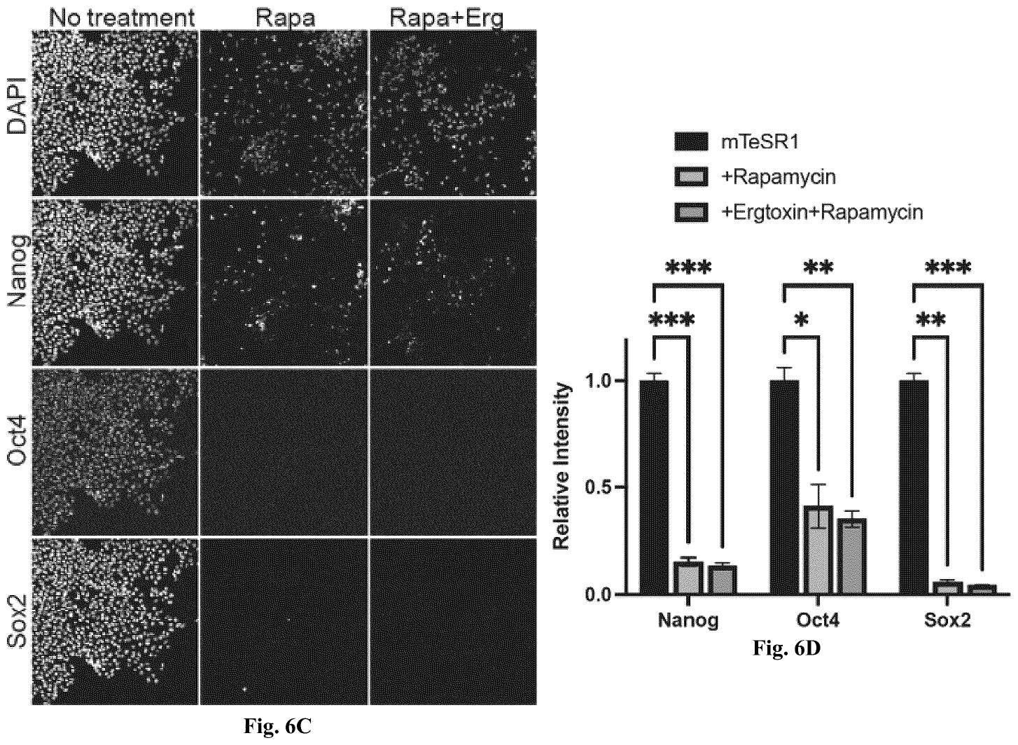

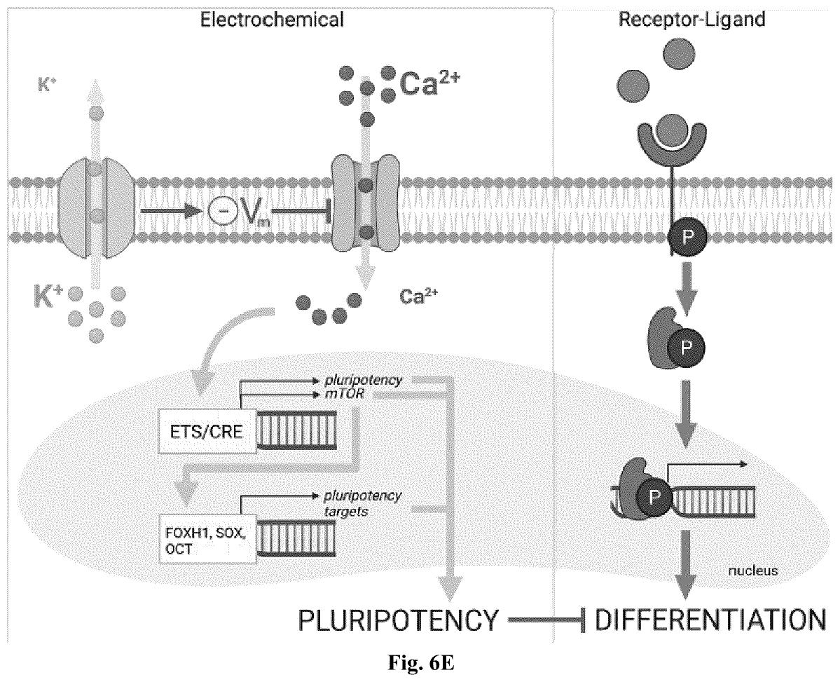

Finally, the present study tested whether the findings herein would also apply to human embryonic stem cells (hESCs). At stage 9, Xenopus animal cap cells are pluripotent in that they can, under appropriate conditions, form derivatives of any of the three germ layers (FIGS. 2A-20). However, they contrast with hESCs in their limited capacity for self-renewal as the brisk pace of embryonic development proceeds. Therefore, the present study turned attention to hESCs to test the findings in the context of a self-renewing pluripotent state and to determine their relevance to human development. hESC are already highly pluripotent, and it was wondered if depolarization would lead to elevations in the pluripotency markers OCT4 and SOX2. Indeed, hESCs grown for two days with Ergtoxin to specifically block KCNH channels showed a modest but significant elevation of these markers over their already high levels in the pluripotency state as indicated by immunostaining and qPCR against these markers (FIGS. 6A, 6B, 18A and 18B). While not as specific as Ergtoxin for KCNH channels, Barium showed similar trends but did not rise to statistical significance. qPCR for the markers OCT4, SOX2 and NANOG also revealed upregulation of these genes at the transcript level, with OCT4 and SOX2 upregulated by day 2 and all three genes upregulated on day 5 (FIGS. 18A-18F).

The present study also tested whether blocking K⁺ channels with Ergtoxin affected the kinetics of differentiation. BMP4 induces differentiation to either mesodermal or extraembryonic fates in a dose-dependent manner. Ergtoxin caused a significant delay in downregulation of pluripotency markers such as SOX2 at 12 hours with a similar trend in NANOG (FIGS. 18C and 18D). As in Xenopus, the timing of differentiation of human embryonic stem cells appears significantly affected under depolarizing conditions.

To test whether the role of mTOR signaling downstream of membrane depolarization is conserved, the present study treated hESCs with rapamycin with or without Ergtoxin. Treatment with rapamycin led to reduction of pluripotency markers in a dose dependent manner with near complete loss by 5 days (FIG. 6C). In the presence of rapamycin, Ergtoxin had no effect on pluripotency markers (FIG. 6D), indicating that mTOR is downstream of membrane depolarization in hESCs as in Xenopus. Although rapamycin did reduce the final cell numbers per well, it inhibited the effect of Ergtoxin on pluripotency marker expression independently of the density at which cells were seeded (FIG. 18E) and of the final cell number in the well (FIG. 18F). Taken together, the data herein support that the polarization of membrane potential via KCNH channels promotes the exit from pluripotency and the activation of differentiated cell fates in Xenopus and human cells.

### Example 2-10

A model in which membrane voltage regulates intracellular calcium during a critical stage of embryonic development, at which point cells need to extinguish pluripotency factors in order to activate a program of cellular differentiation, is proposed herein (FIG. 6E). The K⁺ and Ca′ channel network upstream of pluripotency factor expression potentially represents an extremely robust control mechanism over the first stages of organism development: ion channels are effective at low expression levels, modular and thus partially redundant with respect to each other (i.e. subunits of different channels can heterodimerize to form a channel if subunits of the same channel are unavailable), and their function depends on the existence of a simple ion concentration gradient across the plasma membrane. The basic elements of this regulation, K⁺ and Ca′, are readily available extracellularly, conferring this system with some independence from protein-dependent cell signaling. Ultimately, ion channel networks may represent an additional mechanism to regulate cell fate during development, which is complementary to the established paradigm of gene expression regulation by secreted factors and ligand-receptor signaling (FIG. 6E).

The present study showed that regulation of voltage gated calcium channels by Vₘ is critical for the exit from pluripotency. Based on this, it is suggested that low intracellular calcium reduces the expression of mTOR and pluripotency factors, which is conducive to differentiation onset.

The work herein connects Vₘ and intracellular calcium as upstream of this pluripotency program. The work herein demonstrates the importance of Vₘ in vivo during early embryonic development as well as in vitro in human stem cells. Importantly, this pathway is readily manipulated by a wide range of highly specific channel inhibitors or simple changes in extracellular ionic concentrations. Therefore, the present study defines multiple tools for pluripotency manipulations in embryos, organoids, and adult tissues where stem cells play a critical role.

### Example 2-11: Methods

### Xenopus Husbandry

Adult Xenopus tropicalis were raised and housed according to the established protocols which were approved by the Yale Institutional Animal Care and Use Committee. The present study induced ovulation, performed IVF, and raised embryos in 1/9×MR. The present study staged X. tropicalis embryos according to Nieuwkoop and Faber (Normal table of Xenopus laevis (Daudin): a systematical and chronological survey of the development from the fertilized egg till the end of metamorphosis. (Garland Pub., 1994))

Morpholino Oligonucleotides, mRNA and CRISPRs

All injections of Xenopus embryos were performed at the one-cell stage using a fine glass needle and Picospritzer system. A kcnh6 translation blocking (kcnh6 MO, 5′-GGTCCTCGAAGTTTAGGATAAACAT-3′, SEQ ID NO:1) and a scrambled morpholino oligonucleotide were obtained from Gene Tools LLC and injected at 10 ng to deplete kcnh6 or as a control, respectively. CRISPR sgRNAs for kcnh6 targeted either exon 3 or exon 4 based on the v7.1 gene model of the X. tropicalis genome (CRex3: 5′-GGAATAAGGGGTGAAGACAGCGG-3′, SEQ ID NO:2 and CRex4: 5′-AGGGCGCTCTACATTTCCAATGG-3′, SEQ ID NO:3). CRISPR sgRNAs for cacna1c (5′-GCAGACGGGGGCAGCGCCATTGG-3′, SEQ ID NO:4) and cacna1g (5′-GGTTAATGGCTCTCAGCGGGCGG-3′, SEQ ID NO:5) were designed from the v7.1 model of the Xenopus tropicalis genome. For F0 CRISPR knockdown, embryos were injected with 1.5 ng Cas9 Protein (PNA-Bio) and 400 pg of targeting sgRNA and raised to desired stages. For pitx2 and coco expression analyses, the dose of kcnh6 sgRNA was halved to a subphenotypic dose of 200 pg to obtain embryos without gross morphological gastrulation defects. Full length human KCNH6 (NM 030779.3; cloned in pCS107), GCaMP6 (subcloned in pCSDest) and mCherry cDNAs (Addgene #34935; in pCS2+), were used to generate capped mRNAs in vitro by first linearizing with appropriate restriction enzymes and then transcribing with the mMessage machine kit (Ambion). mRNAs were injected at 3 pg (human KCNH6), 150 pg (GCaMP6) and 150 pg (mCherry) per embryo. Embryos were raised at 21° C. to allow time for sufficient expression levels at blastula/gastrula stages.

### Inference of CRISPR Edits (ICE) Analysis

Genomic DNA from CRISPR and control embryos were obtained by lysing individual, stage 45 tadpoles in 50 mM NaOH and amplifying PCR fragments around the CRISPR target site that encompass approximately 200 bp upstream and 500 bp downstream of the site. The following primers were used for CRISPRs targeting exons 3 and 4 of the kcnh6 locus, respectively: CRex3-F: 5′-CAGGACTGATGAAAGCAAGC-3′ (SEQ ID NO:6) and Crex3-R: 5′-GCTTATCCATAGCTGTAACAACG-3′ (SEQ ID NO:7); CRex4-F: 5′-GAGACAGTAGGCTGTTCC-3′ (SEQ ID NO:8) and CRex4-R: 5′-CCACAAGCAGTTTCACTACC-3′ (SEQ ID NO:9). PCR fragments were Sanger sequenced using the same forward primers, and sequencing traces were uploaded for analysis with the Synthego ICE analysis web tool to assess editing outcomes.

### Organ Situs

Stage 45 Xenopus embryos were paralyzed with benzocaine or tricaine and scored with a light stereomicroscope. Cardiac looping was determined by position of the outflow tract; D-loop: rightward, L-loop: leftward; A-loop: midline. Normal intestinal looping was scored as counter-clockwise rotation of the gut, while abnormal intestinal looping was scored as completely inverse gut rotation (clockwise) or complete lack of looping (un-looped). While a completely inverted gut rotation is clearly an abnormality of LR patterning, an unlooped gut is less clear so the present study only considered an unlooped gut as abnormal situs when combined with abnormal placement (left-sided or midline) of the gall bladder. To quantify total abnormal organ situs, each tadpole was counted only once, regardless of whether multiple organs were affected.

### Whole Mount In Situ Hybridization

Digoxigenin-labeled antisense probes for pitx2 (TNeu083k20), dand5/coco (TEgg007d24), myoD (Tneu017H11), myf5 (TGas127b01), tbxt (TNeu024F07), foxj1 (Tneu058M03), ectodermin (TNeu104j16), foxIla (Tgas002H16), mixer (TGas105b05), vegT (TGas066f22), gsc (TNeu077f20), xnr3 (Tgas011k18), vent2 (BG885317), oct25 (TGas051h05), oct60 (IMAGE: 7526158), oct91 (IMAGE: 7575764), vent1 (BG487195), sox2 (Tgas061h22), cytokeratin (IMAGE:6991625) and sox1713 (BG886038) were in vitro transcribed using T7 High Yield RNA Synthesis Kit (E20405) from New England Biolabs. In order to generate a full-length antisense probe for X. tropicalis kcnh6, kcnh6 cDNA was cloned from stage 45 tadpole whole mRNA using primers xtkcnh6-F: 5′-ATGTTTATCCTAAACTTCGAGGACC-3′ (SEQ ID NO:10) and xtkcnh6-R: 5′-CTAACTTCCTGGAAGACCTGGG-3′ (SEQ ID NO:11) (XM_012952904.1). It was noted that kcnh6 had been misannotated as kcnh2 in the v7.1 model of the X. tropicalis genome (The present study used NCBI Annotation XP_012808358.2 to identify KCNH6). Embryos were collected at the desired stages, fixed in MEMFA for 1-2 h at room temperature (RT) and dehydrated in 100% ethanol. GRPs were dissected post fixation and prior to dehydration to detect dandy. To detect putative gene expression in the prospective endoderm (mixer, vegT, kcnh6) gastrula stage embryos were bisected to facilitate better probe access. Briefly, whole mount in situ hybridization of digoxigenin-labeled antisense probes was performed overnight, the labelled embryos were then washed, incubated with anti-digoxigenin-AP Fab fragments (Roche 11093274910), and signal was detected using BM-purple (Roche 11442074001).

### Medium Conditions and Treatments of Embryos

Normal embryonic medium is 1/9× modified Ringer's (MR) containing 11 mM NaCl, 0.2 mM KCl, 0.2 mM CaCl₂), 0.1 mM Mg₂Cl and 0.55 mM HEPES. To allow Ergtoxin to penetrate the embryos, the present study manually removed the vitelline envelope of stage 8 embryos and incubated embryos in 1/9×MR containing 50 nM Ergtoxin (Alomone STE-450) until stage 12. Embryos were then transferred back into 1/9×MR lacking Ergtoxin to develop until stage 45 in order to score organ situs. Barium chloride was applied into the medium at 20 mM and embryos were thoroughly rinsed in 1/9×MR after each incubation period for further development in Ba2+-free medium. For extracellular K⁺ manipulations, the KCl concentration in 1/9×MR was modified from 0.2 mM (normal) to 20 mM (high) or 0 mM (low). The ionophore valinomycin (ACROS) and L-type VGCC blocker Nifedipine (ACROS) were diluted in DMSO as stock solutions and applied to embryos in 1/9×MR at 2 nM and 10 μM respectively. Treatments performed during gastrulation were applied from stage 8 through stage 12, and embryos were then rinsed thoroughly and returned into 1/9×MR. For rapamycin, the present study created a standard stock solution of 50 mg/ml in DMSO. The stock solution was diluted 1:2500 in the appropriate embryonic media (final 20 μg/ml). Embryos were treated at stage 7 and then fixed at stage 11 for in situ hybridization.

### GRP Immunofluorescence

Embryos were fixed at stage 17 in 4% paraformaldehyde-PBS for 2 h at RT, washed in PBS, and then dissected to obtain GRPs. GRPs were permeabilized for 30 min at RT using 0.1% Triton-PBS (PBST), then blocked in 1% BSA-PBST for 1 h at RT and incubated in primary antibodies diluted in 1% BSA-PBST overnight at 4° C. (anti-myoD LsBio C143580-100 or anti-acetylated tubulin Sigma T-6793). GRPs were then washed in PBST for 30 min and then incubated with secondary antibodies in 1% BSA-PBST for 1h at RT. Phalloidin (1:50; Molecular Probes) and Hoechst 33342 (1:1000; Molecular Probes) were diluted into the secondary antibody solution. Images were acquired using a ZEISS 710 laser scanning confocal microscope.

### Intracellular Vₘ Recordings

For recordings, devitellinized, stage 10 kcnh6 or control MO injected embryos were mounted into non-toxic clay with their animal pole exposed and covered with 1/9×MR. To investigate the resting potential, animal pole cells were impaled with a high-impedance (˜70 Me), sharp microelectrode filled with 3 M KCl for intracellular recordings. The recordings were made using an Axon 200B amplifier and digitized using a Digidata 1320 digitizer. Jclamp software for Windows was used in current clamp mode. All electrodes were zeroed just before entry into the cells.

For the series of intracellular recordings in high K⁺ and choline treated embryos, stage 8-9 embryos were impaled similarly with an electrode of −40 Me. These recordings were made using a HEKA EPC10 amplifier. The present study used HEKA PatchMaster v2x67 software for Windows. All electrodes were zeroed just before entry into the cells.

### Calcium Imaging

GCaMP6 and mCherry mRNAs were mixed and injected into embryos at the one-cell stage. Half of these embryos were then injected with kcnh6 MO and the other half with control MO, still at the one-cell stage. Embryos were transferred at stage 10 into the round wells of a press-to-seal silicone isolator (Sigma 53685) mounted between two cover slips in 2% Methylcellulose- 1/9×MR. GcaMP6 and mCherry fluorescence was then captured for 20s (1 frame per second) via time lapse in the whole animal pole of each embryo with a 20× objective of an LSM710 confocal microscope using identical acquisition settings across Control MO and kcnh6 MO embryos. Time lapse recordings were conducted randomly and in an unbiased manner in regard to presence and intensity of calcium transients. However, all embryos did display transient increases in GcaMP6 fluorescence, varying in intensity and spreading to multiple cells. The frames of each recording were sorted to identify the calcium transient peak (in area), and GcAMP6 fluorescence intensity was quantified at peak as a ratio to mCherry in mCherry+ cells. The maximum Ca′ transient area was calculated by demarcating in Fuji the GCaMP6(+) vs GcaMP6(−) area of the animal pole at transient peak. To avoid mosaicism artifacts, only embryos with even, non-mosaic mCherry expression across the entire animal pole were considered. To avoid embryonic stage dependent fluctuations in Ca′ transient size, the present study verified each embryo for stage by progression of blastopore closure and alternated recordings of control and kcnh6 MO embryos. Of note, there were no notable differences in mCherry expression between Control MO and kcnh6 MO embryos.

### Animal Cap Pluripotency Assays

After manually removing the vitelline envelope of stage 9 or 12 embryos, animal caps were excised and placed on agarose coated dishes in 1/9×MR solution. Caps were then directly placed into agarose coated wells of a 96-well plate in ⅓×MR containing 0.1% BSA and cultured without activin to allow for differentiation into epidermis, with low (20 ng/ml) activin to induce mesoderm, or high activin (200 ng/ml) to induce endoderm. Explants were raised at 25° C. until reaching the equivalent of stage 18 (monitored in whole embryos of the same batch), then fixed in 4% paraformaldehyde, washed in PBS, bleached to eliminate pigmentation (0.5×SSC, 5% formamide, 1.2% H₂O₂), and then processed by in situ hybridization as described above.

### RNA-Seq

For RNA-Seq, embryos were kept at 25° C. either in 1/9×MR or in 10 mM KCl solution, and 10 embryos were harvested per time point and condition every 30 min starting at stage 8 and concluding at stage 13. Samples were immediately frozen and kept at −80° C. until homogenized in 100 μl Trizol spiked with ERCC RNA Spike-In Mix. 10 μl ERCC RNA Spike-In Mix (Thermo Fisher Scientific) were first diluted into a final volume of 870 μl DEPC water and then further diluted 1:10 into Trizol, which was used to homogenize the samples. Total RNA was purified from the embryo Trizol homogenates according to the manufacturer's recommendations. After isopropanol precipitation, RNAs were resuspended in DEPC water and any contaminating genomic DNA was removed by overnight precipitation in 5M LiCl at 4° C. RNA was subsequently pelleted and washed twice with 70% ethanol. All RNAs were resuspended in DEPC water (2 μl/embryo), and finally, RNA quality was verified by Bioanalyzer. All libraries were sequenced with 100-bp paired-ends on an Illumina NovaSeq6000.

### Xenopus Biological Replicates, Statistical Methods, Graphs and Models

In experiments where embryos were evaluated for phenotypes and scored (gastrulation, left-right patterning, in situ hybridizations) the present study carried out three to five biological replicates and Fisher's exact test to evaluate statistical significance. The animal cap experiment was performed twice with a total score of four to eight animal caps per experiment. For the calcium transient analyses, data was collected from three to five embryos in each experiment in three independent experiments, and statistical analyses on GCaMP/mCherry fluorescence intensity as well as Ca²⁺ transient area were performed using student's t-test. For whole cell electrophysiological recordings, three to five embryos (two cells each) were examined for their membrane potential and statistical significance was tested by student's t-test. Graphs were designed using GraphPad Prism software. Models were created with BioRender.com.

hESC Culture

hESCs were grown in mTeSR1 (STEMCELL Technologies) in tissue culture dishes coated with Matrigel (Corning; 1:200 in DMEM/F12) and kept at 37° C., 5% CO2. The cell lines used were ESI017 (ESIBIO) and H9. Cells were routinely passaged using dispase (STEMCELL Technologies) and tested for mycoplasma contamination and found negative. For rapamycin experiments, cells were grown in MEF-conditioned HUESM media supplemented with 20 ng/ml bFGF with or without 100 nM rapamycin, which the present study found to increase the survival of rapamycin treated cells compared to cells grown in mTeSR1.

hESC Treatments and Differentiation

Cells were dissociated with accutase and seeded onto 8 well imaging slides (ibidi 80826) at a density of 4-6×10⁴/cm². Cells were seeded and maintained in Rock-inhibitor Y27672 (MCE; 10 μM) to increase survival and the uniformity of response. Treatments with 1 mM BaCl₂ or 10 or 25 nM Ergtoxin or 100 nM rapamycin were initiated 4 hours after seeding. Differentiation was initiated 24 hours after seeding where indicated. To initiate differentiation, the media was replenished with/without BaCl₂ or Ergtoxin and treated with the indicated growth factors or small molecules. Cells were incubated for the indicated times without media change before fixation.

Immunofluorescence of hESCs

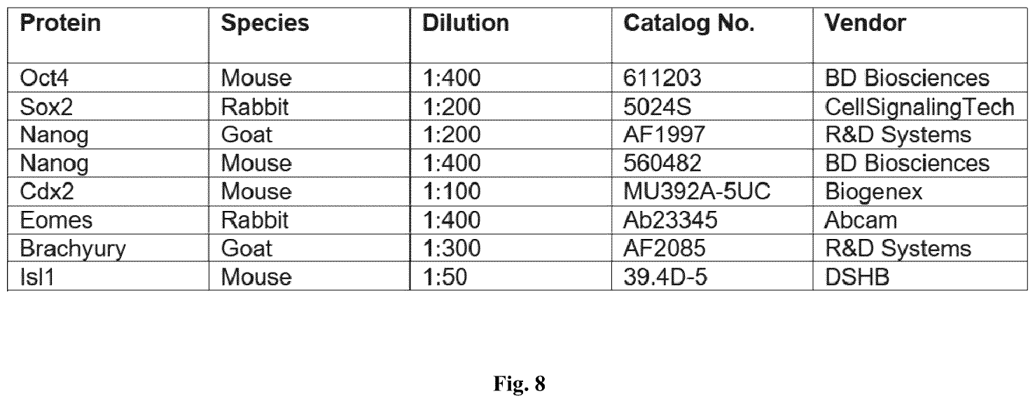

Cells were fixed for 30 min in 4% paraformaldehyde, rinsed twice with DPBS (without Ca²⁺ and Mg²⁺, denoted DPBS^{-/-}), and blocked for 30 min at room temperature. The blocking solution contained 3% donkey serum and 0.1% Triton X-100 in 1×DPBS^{-/-}. After blocking, the cells were incubated with primary antibodies at room temperature for 2 hours. Antibodies and concentrations are listed below. Cells were washed three times with DPBST (1×DPBS^{-/-} with Tween 20) and incubated with secondary antibodies (AlexaFluor 488 A21206, AlexaFluor 555 A31570 and A21432, and AlexaFluor 647 A31571, Thermo Fisher; 1:500) and DAPI for 30 min at room temperature. After secondary antibody incubation, samples were washed in DPBST and then DPBS at room temperature. The antibodies used for the experiments are also listed in FIG. 8.

hESC Imaging and Analysis

Images were acquired using a 20×, NA 0.75 objective on an Olympus IX83 inverted epifluorescence microscope or an Olympus/Andor spinning disk confocal microscope. Cell segmentation was performed using ilastik software. This segmentation was cleaned (to remove debris and to separate merged cells) and mean nuclear protein intensities as well as standard errors were quantified using a custom MATLAB code. Nuclear intensities were normalized by DAPI to correct for intensity variation due to optics. Code is available at https://github.com/warmflasha/celltracker.

qPCR

For qPCR, hESCs were grown with or without ErgToxin (25 nM) for the indicated times. RNA collection and DNase treatment were performed using the RNAqueous®-Micro Total RNA Isolation Kit (AM1931) and cDNA was synthesized with the SuperScript Vilo cDNA Synthesis Kit (Fisher Scientific 11754-050). qPCR measurements were collected using SYBR Green reagent (LifeTech-4367659) on a Step OnePlus instrument (Applied Biosciences). Data were normalized using the housekeeping gene GAPDH. Primers for qPCR were: OCT4: 5′-caagctcctgaagcagaagag-3′ (SEQ ID NO:12), 5′-ccaaacgaccatctgccgcttt-3′ (SEQ ID NO:13), SOX2: 5′-ccatgcaggttgacaccgttg-3′ (SEQ ID NO:14), 5′-tcggcagactgattcaaataata-3′ (SEQ ID NO:15), NANOG: 5′-tgggatttacaggcctgagcca-3′ (SEQ ID NO:16), 5′-aagcaaagcctcccaatcccaaa-3′ (SEQ ID NO:17), GAPDH: 5′-caccgtcaaggctgagaacg-3′ (SEQ ID NO:18), 5′-gccccacttgattttggagg-3′ (SEQ ID NO:19).

### Quantification RNA-Seq

Stranded paired end 100 bp RNA-seq reads were aligned to the Xt9.1 genome combined with ERCC spikes using STAR1 and quantified as transcripts per million (TPM) for each isoform with RSEM2 using the RSEM-STAR pipeline, with additional options “—seed 1618 —calc-pme —calc-ci —estimate-rspd —paired-end”. Using the ERCC spikes the present study identified a batch-dependent GC bias where AT-rich transcripts were preferentially lost as compared to GC-rich transcripts (FIG. 18B). The present study leveraged knowledge of spike-in concentrations to build a GC model correction based on the dinucleotide content of RNAs. The present study calculate the propensity of each of the 16 dinucleotides (AA, AC, . . . , TG, TT) within each spike sequence, with fᵢₖ is the frequency of dinucleotide k within sequence i, its propensity is pᵢₖ=fᵢₖ/Σfᵢⱼ. The present study then employed the following linear model to correct the TPM tsi of RNA spike i in sample

s to its known concentration cᵢ:

$$log {c}_{i} = α + {β}_{T} log {t}_{si} + {\sum }_{j} {β}_{j} log {ρ}_{ij}$$

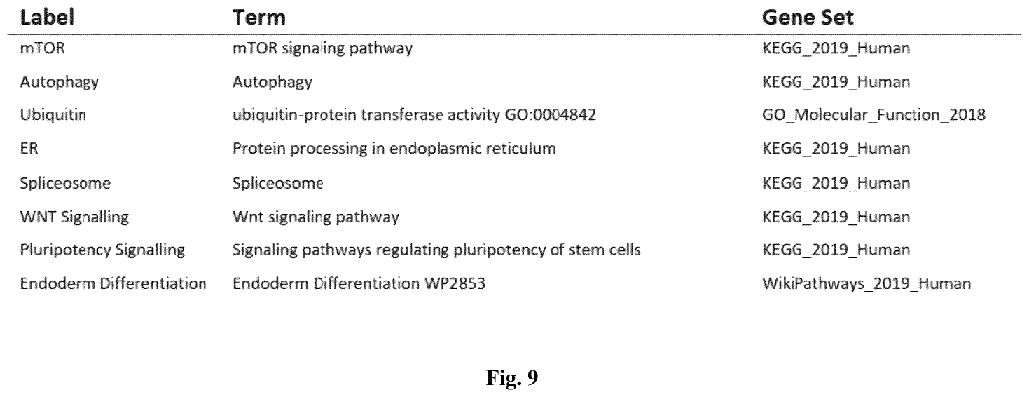

The present study used the GLM.jl (https://github.com/JuliaStats/GLM.jl) in the Julia language to apply this model and add a pseudocount of 2 to all dinucleotide frequencies. As the GC effect varies between UIC and high K⁺ samples (Ext Data FIG. 9b), the present study applied the correction independently to UIC and High K⁺. The correction is able to explain a significant proportion of variance in spike TPM, increasing R2 from 0.807 and 0.733 to 0.965 and 0.964 respectively from UIC and high K⁺ samples. The present study applied this correction to each isoform of all genes quantified with the dinucleotide propensities of each isoform and RSEM isoform quantifications. The present study then sumed all corrected quantifications at the isoform level to derive gene level quantifications. This allows us to account for differing isoforms of the same gene with differing dinucleotide propensities.

### Filtering of Genes for Differential Expression Analysis

The present study first filtered 34,192 quantified genes to find those with sufficient temporal expression for further analysis, the present study selected genes that had runs of 6 consecutive samples with uncorrected TPM>0.4. This resulted 13,310 from which the present study excluded a further 162 genes which where excessively altered by the above-described correction procedure, these had log 2 fold changes between corrected or uncorrected quantifications outside of the interval (−2.5, 4.5). After dinucleotide correction and filtering the present study found excellent concordance between samples, with minimal evidence of outlying samples, by Spearman Correlation comparisons and principal components analysis (PCA) (FIGS. 18C and 18D). The two domains in visible in pairwise Spearman comparisons (FIG. 18C) reflect the loss of maternal RNA and the commencement of widespread zygotic transcription. Projection onto the first two principal components revealed that samples lie in appropriate order on a trajectory in 2D space, and the largest divergences between UIC and high K⁺ occur midway through the time series in agreement with Gaussian process differential expression and clustering described below. Corrected dinucleotide gene expression abundances are used in all analyses.

### Temporal Differentiation Expression

To determine genes temporally differentially expressed the present study used Gaussian process (GP) regression. All GP regression was performed with GaussianProcesses.j1 (https://github.com/STOR-i/GaussianProcesses.j1; https://arxiv.org/abs/1812.09064). Due to the overdispersed nature of RNA-seq count data, the present study applied a variance stabilising transform that puts all genes on the same scale: yₛᵢ=\sqrt{α+βxₛᵢ/mᵢ}, with xₛᵢ the dinucleotide corrected abundance of gene i in sample s, mᵢ the maximum xₛᵢ over all samples, and α=1, β=1000. The present study then performed exact GP regression (GP prior and a Gaussian likelihood) with Matern52 kernel, the present study optimized the three associated hyperparameters: σ_{f}² or the signal variance, τ the timescale (this parameter is commonly referred to as the lengthscale [symbol] ), and σ_{f}² the sample noise variance. Parameters are selected by optimising marginal log-likelihood with parameters in log space: log σ_{f}, log τ, log σₙ, and to ensure physiologically reasonable values for each the present study placed Gaussian priors, [symbol] (μ, σ) over each of these variables respectively [symbol] (1.4, 4.0), [symbol] (1.2, 1.0), [symbol] (1.0, 0.75). Finally, the present study reported GP median and 95% confidence intervals through the inverted data transformation \hat{x}ₛᵢ=mᵢ(ŷₛᵢ¹−α)/β and set \hat{x}ₛᵢ=0 for ŷₛᵢ<\sqrt{α}.

To determine temporal differential expression, the present study calculated a marginal likelihood ratio for whether separate GP models for UIC and high K⁺ or a single GP model for all data combined is preferred. If Lᵤ and Lₖ are the marginal log-likelihoods for UIC and high K⁺ respectively, and Lᵤₖ is the marginal log-likelihood for a single regression through UIC and high K⁺ together. Then the present study calculate log-likelihood ratio L_{R}=Lᵤ+Lₖ−Lᵤₖ of evidence in favour of two models (essentially that the UIC and High K⁺ have different expression trajectories for a given gene) and determine genes with LR>0 as temporally differentially expressed. This resulted in 5144 differentially expressed genes, with 4043 activated and 1101 repressed (FIG. 18D which shows that the max absolute divergence z-score between UIC and high K⁺ trajectories increase with LR). The present study also considered a more stringent condition for differential expression using the Bayesian Information Criterion (BIC)3, that resulted in 2388 differentially genes. It was found that this diminished differential expression gene set enrichments described below, indicating that the BIC was too conservative and the present study continued with the condition based on log-likelihood ratio.

### Clustering

To determine sets of differentially expressed genes with similar trajectories, the present study applied K-means clustering to activated and repressed genes independently. The present study define a gene as activated if Gaussian process median for High K⁺ exceeds UIC on average, and repressed if it does not, the present study found no genes for which the mean of High+ and UIC differences was zero. The present study cluster UIC and High K⁺ genes by taking Gaussian process medians and normalising by the maximum value experience by UIC or High K⁺. The present study then clustered both trajectories together employing the kmeans function offered by Clustering.jl (https://github.com/JuliaStats/Clustering.jl) with default settings and random seed 16. To select the cluster number, the present study calculated the silhouette score for activated and repressed clusters for k=2-10. The present study found that the maximal mean silhouette score activated genes was k=3 and for repressed genes was k=2, but that scores were broadly similar for k=2-4 and decreased significantly for k>4, suggesting that k=4 provides a reasonable partition of the data. In line with this the present study explored the clusters from k=2-10, and found that key clusters were not well-resolved for k<4 and that k>4 clusters refined k=4 behaviours. As k>4 did not reveal new behaviours and did not improve gene set enrichments, the present study selected k=4 to cluster activated and repressed genes.

### Gene Set Enrichments

To assess the composition of each cluster the present study performed gene set enrichments using Enrichr. The present study took genes from each cluster with a known Xenopus gene symbol and converted these to human symbols, by removing any “.N” suffix for an integer N (for example, ventx1.1 becomes ventx1) and converting to uppercase. The present study then made the following substitutions to convert certain known Xenopus gene symbols to human where the name of the ortholog has diverged or only a paralog exists: pou5f3→POU5F1, mix1→mixl1, dppa2→DPPA4, lefty→lefty2, ventx1-3→NANOG, mespb→MESP1, sox17a/b→SOX17. The present study removed any duplicate names that arose in this process. The present study calculated enrichments for the following gene sets: KEGG_2019 Human, BioPlanet_2019, WikiPathways_2019 Human, GO_Biological_Process_2018, GO_Molecular_Function_2018, GO_Cellular_Component_2018, ChEA_2016. The present study calculated enrichments for each cluster individually and consecutive combinations of the 4 clusters: 1, 2, 3, 4, 12, 23, 34, 123, 234, 1234. Selected enrichments are given in FIG. 4C, and terms are given shortened labels for brevity in FIG. 9.

### Motif Analysis

To find motifs enriched in the promoters, the present study took the 500 bp upstream of the promoter of the maximally expressed isoform for each gene in the four activated and four repressed clusters, along with a background of the 500 bp upstream of all annotated TSS in the Xt9.1 genome. The present study extracted fasta files for each of these sets of regions, and then used findMotifs.pl from Homer5 to search for known motifs with options: “findMotifs.pl clusterAB.fa fasta outAB -fasta background.fa -nomotif” where A∈{activated, repressed} and B∈{1, 2, 3, 4}. The present study filtered results to select best matching motifs from related families, namely the present study collapsed all ETS motifs to the canonical Homer ETS promoter motif; all SP and KLF motifs to SP1; SOX motifs to SOX2; all HOX motifs to HOXD13 (the highest scoring HOX); the present study represented all GFY and Ronin matches as ZNF143 (for which the motifs overlap); and the present study excluded motif annotated as PRDM10, due to low confidence in the motif. The motif annotated as ATF1 is an example of the cAMP response element (CRE) bound by CREB factors including ATF1, the present study labels this as CRE/ATF1. The top 16 motif enrichments are given in FIG. 18H, in FIG. 4D the present study gives the top 6 motif matches, excluding ZNF143 due to divergence from the JASPAR database, to which the present study adds CRE/ATF1 as a putative calcium responsive element motivated by CREM/CREB1 gene set enrichments (FIG. 18J).

To calculate CRE and ETS motif enrichment for mTOR and pluripotency genes, the present study took genes annotated with the terms mTOR signaling pathway and Signaling pathways regulating pluripotency of stem cells from KEGG_2019 Human as provided by Enrichr4 that are activated in high K⁺ (LR>0) and are present in clusters A1 and A2. The resulting genes were subjected to the same promoter analysis, using Homer to calculate the occurrence of the maximal ATF/CRE family motif and the ETS motif in these promoters and the background set to report Fisher Exact test p-values and Odds Ratios.

### Enumerated Embodiments

In some aspects, the present invention is directed to the following non-limiting embodiments:

Embodiment 1: A method of maintaining pluripotency in a cell, comprising at least one of the following: reducing the membrane potential of the cell; activating a voltage gated calcium channel on the plasma membrane of the cell; or increasing the calcium ion concentration in the cell.

Embodiment 2: The method of Embodiment 1, wherein reducing the membrane potential of the cell comprises at least one of the following: subjecting the cell to an extracellular environment having a high concentration of potassium ions; inhibiting a potassium channel on the plasma membrane of the cell; or contacting the cell with a potassium selective ionophore.

Embodiment 3: The method of any one of Embodiments 1-2, wherein reducing the membrane potential of the cell comprises subjecting the cell to an extracellular environment having a concentration of potassium ions of about 0.5 mM or higher, such as about 0.75 mM or higher, about 1 mM or higher, about 2 mM or higher, about 3 mM or higher, about 4 mM or higher, about 5 mM or higher, about 7.5 mM or higher, about 10 mM or higher, about 12.5 mM or higher, about 15 mM or higher, about 20 mM or higher, about 25 mM or higher, about 30 mM or higher, about 40 mM or higher, or about 50 mM or higher.

Embodiment 4: The method of any one of Embodiments 1-2, wherein reducing the membrane potential of the cell comprises inhibiting a potassium channel on the plasma membrane of the cell, and wherein the potassium channel comprises an inwardly-rectifying voltage gated potassium channel.

Embodiment 5: The method of Embodiment 4, wherein the inwardly-rectifying voltage gated potassium channel comprises potassium voltage-gated channel subfamily H member 6 (KCNH6).

Embodiment 6: The method of any one of Embodiments 2 and 4-5, wherein inhibiting the potassium channel on the plasma membrane of the cell comprises contacting the cell with a potassium channel inhibitor.

Embodiment 7: The method of Embodiment 6, wherein the inhibitor of the potassium channel comprises barium ions or an Ergtoxin.

Embodiment 8: The method of any one of Embodiment 1-2, wherein reducing the membrane potential of the cell comprises contacting the cell with a potassium selective ionophore comprising valinomycin, BME 44 (2-Dodecyl-2-methyl-1,3-propanediyl bis[N-[5′-nitro(benzo-15-crown-5)-4′-yl]carbamate]), or BB15C5 (Bis[(benzo-15-crown-5)-4′-ylmethyl] pimelate).

Embodiment 9: The method of Embodiment 1, wherein the voltage gated calcium channel is an L-type calcium channel or a T-type calcium channel.

Embodiment 10: The method of any one of Embodiments 1-9, wherein the cell is a stem cell.

Embodiment 11: The method of any one of Embodiments 1-10, wherein the cell is an embryonic stem cell.

Embodiment 12: The method of any one of Embodiments 1-11, wherein the cell is in an organism, a cultured primary cell, or a cultured cell line.

Embodiment 13: The method of any one of Embodiments 1-12, wherein the cell is from a vertebrate origin, optionally from a mammalian origin and/or a human origin.

Embodiment 14: A composition, comprising: a culture medium; and a pluripotent cell, wherein potassium ions in the culture medium are present in a concentration of about 0.5 mM or higher, and wherein a pluripotency of the pluripotent cell is maintained by the concentration of potassium ions in the culture medium.

Embodiment 15: The composition of Embodiment 14, wherein the concentration of the potassium ions in the culture medium reduces the membrane potential of the cell, thereby maintaining the pluripotency of the pluripotent cell.

Embodiment 16: The composition of any one of Embodiments 14-15, wherein the culture medium comprises: at least one inorganic ion selected from a sodium ion, a potassium ion, a calcium ion, a magnesium ion, a chloride ion, a sulfate ion, a carbonate ion, a bicarbonate ion, a phosphate ion, a phosphate monobasic ion, a phosphate dibasic ion; an amino acid; and a vitamin.

Embodiment 17: The composition of claim 16, wherein the amino acid comprises at least one selected form the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine; the vitamin comprises at least one selected from the group consisting of pantothenate, choline, folic acid, inositol, nicotinamide, pyridoxine, riboflavin, and thiamine; the culture medium further comprises a carbohydrate; or the culture medium further comprises one or more selected form the group consisting of pyruvate, lipoic acid, biotin, a buffering agent, and a pH indicator.

Embodiment 18: The method of any one of Embodiments 14-17, wherein the pluripotent cell is a stem cell or an embryonic stem cell.

Embodiment 19: The method of any one of Embodiments 14-18, wherein the pluripotent cell is from a vertebrate origin, a mammalian origin, or a human origin.

Embodiment 20: The method of any one of Embodiments 14-19, wherein the pluripotent cell does not have the cell potency to develop in to a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method of maintaining pluripotency in a cell, comprising at least one of the following:
reducing the membrane potential of the cell;
activating a voltage gated calcium channel on the plasma membrane of the cell; or
increasing the calcium ion concentration in the cell.

2. The method of claim 1, wherein reducing the membrane potential of the cell comprises at least one of the following:
subjecting the cell to an extracellular environment having a high concentration of potassium ions;
inhibiting a potassium channel on the plasma membrane of the cell; or
contacting the cell with a potassium selective ionophore.

3. The method of claim 1, wherein reducing the membrane potential of the cell comprises subjecting the cell to an extracellular environment having a concentration of potassium ions of about 0.5 mM or higher.

4. The method of claim 1, wherein reducing the membrane potential of the cell comprises inhibiting a potassium channel on the plasma membrane of the cell, and wherein the potassium channel comprises an inwardly-rectifying voltage gated potassium channel.

5. The method of claim 4, wherein the inwardly-rectifying voltage gated potassium channel comprises potassium voltage-gated channel subfamily H member 6 (KCNH6).

6. The method of claim 2, wherein inhibiting the potassium channel on the plasma membrane of the cell comprises contacting the cell with a potassium channel inhibitor.

7. The method of claim 6, wherein the inhibitor of the potassium channel comprises barium ions or an Ergtoxin.

8. The method of claim 1, wherein reducing the membrane potential of the cell comprises contacting the cell with a potassium selective ionophore comprising valinomycin, BME 44 (2-Dodecyl-2-methyl-1,3-propanediyl bis[N-[5′-nitro(benzo-15-crown-5)-4′-yl]carbamate]), or BB15C5 (Bis[(benzo-15-crown-5)-4′-ylmethyl]pimelate).

9. The method of claim 1, wherein the voltage gated calcium channel is an L-type calcium channel or a T-type calcium channel.

10. The method of claim 1, wherein the cell is a stem cell.

11. The method of claim 1, wherein the cell is an embryonic stem cell.

12. The method of claim 1, wherein the cell is in an organism, a cultured primary cell, or a cultured cell line.

13. The method of claim 1, wherein the cell is from a vertebrate origin, a mammalian origin, or a human origin.

14. A composition, comprising:
a culture medium; and
a pluripotent cell,
wherein potassium ions in the culture medium are present in a concentration of about 0.5 mM or higher, and
wherein a pluripotency of the pluripotent cell is maintained by the concentration of potassium ions in the culture medium.

15. The composition of claim 14, wherein the concentration of the potassium ions in the culture medium reduces the membrane potential of the cell, thereby maintaining the pluripotency of the pluripotent cell.

16. The composition of claim 14, wherein the culture medium comprises:
at least one inorganic ion selected from a sodium ion, a potassium ion, a calcium ion, a magnesium ion, a chloride ion, a sulfate ion, a carbonate ion, a bicarbonate ion, a phosphate ion, a phosphate monobasic ion, a phosphate dibasic ion;
an amino acid; and
a vitamin.

17. The composition of claim 16, wherein
the amino acid comprises at least one selected form the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine;
the vitamin comprises at least one selected from the group consisting of pantothenate, choline, folic acid, inositol, nicotinamide, pyridoxine, riboflavin, and thiamine;
the culture medium further comprises a carbohydrate; or
the culture medium further comprises one or more selected form the group consisting of pyruvate, lipoic acid, biotin, a buffering agent, and a pH indicator.

18. The method of claim 14, wherein the pluripotent cell is a stem cell or an embryonic stem cell.

19. The method of claim 14, wherein the pluripotent cell is from a vertebrate origin, a mammalian origin, or a human origin.

20. The method of claim 14, wherein the pluripotent cell does not have the cell potency to develop in to a human.


SEQUENCE LISTING (19 sequences)

SEQ ID NO: 1 - DNA, 25 bp, synthetic construct
  source 1..25: mol_type = other DNA
ggtcctcgaagtttaggataaacat

SEQ ID NO: 2 - DNA, 23 bp, synthetic construct
  source 1..23: mol_type = other DNA
ggaataaggggtgaagacagcgg

SEQ ID NO: 3 - DNA, 23 bp, synthetic construct
  source 1..23: mol_type = other DNA
agggcgctctacatttccaatgg

SEQ ID NO: 4 - DNA, 23 bp, synthetic construct
  source 1..23: mol_type = other DNA
gcagacgggggcagcgccattgg

SEQ ID NO: 5 - DNA, 23 bp, synthetic construct
  source 1..23: mol_type = other DNA
ggttaatggctctcagcgggcgg

SEQ ID NO: 6 - DNA, 20 bp, synthetic construct
  source 1..20: mol_type = other DNA
caggactgatgaaagcaagc

SEQ ID NO: 7 - DNA, 23 bp, synthetic construct
  source 1..23: mol_type = other DNA
gcttatccatagctgtaacaacg

SEQ ID NO: 8 - DNA, 18 bp, synthetic construct
  source 1..18: mol_type = other DNA
gagacagtaggctgttcc

SEQ ID NO: 9 - DNA, 20 bp, synthetic construct
  source 1..20: mol_type = other DNA
ccacaagcagtttcactacc

SEQ ID NO: 10 - DNA, 25 bp, synthetic construct
  source 1..25: mol_type = other DNA
atgtttatcctaaacttcgaggacc

SEQ ID NO: 11 - DNA, 22 bp, synthetic construct
  source 1..22: mol_type = other DNA
ctaacttcctggaagacctggg

SEQ ID NO: 12 - DNA, 21 bp, synthetic construct
  source 1..21: mol_type = other DNA
caagctcctgaagcagaagag

SEQ ID NO: 13 - DNA, 22 bp, synthetic construct
  source 1..22: mol_type = other DNA
ccaaacgaccatctgccgcttt

SEQ ID NO: 14 - DNA, 21 bp, synthetic construct
  source 1..21: mol_type = other DNA
ccatgcaggttgacaccgttg

SEQ ID NO: 15 - DNA, 23 bp, synthetic construct
  source 1..23: mol_type = other DNA
tcggcagactgattcaaataata

SEQ ID NO: 16 - DNA, 22 bp, synthetic construct
  source 1..22: mol_type = other DNA
tgggatttacaggcctgagcca

SEQ ID NO: 17 - DNA, 23 bp, synthetic construct
  source 1..23: mol_type = other DNA
aagcaaagcctcccaatcccaaa

SEQ ID NO: 18 - DNA, 20 bp, synthetic construct
  source 1..20: mol_type = other DNA
caccgtcaaggctgagaacg

SEQ ID NO: 19 - DNA, 20 bp, synthetic construct
  source 1..20: mol_type = other DNA
gccccacttgattttggagg